(19) United States
(12) Patent Application Publication
(10) Pub. No.: US 2007/0191297 A1
(43) Pub. Date: Aug. 16, 2007

(54) METHODS OF TREATING AND MONITORING SYSTEMIC LUPUS ERYTHEMATOSUS IN INDIVIDUALS

(76) Inventors: Matthew D. LINNIK, Solana Beach, CA (US); Tenshang JOH, Encinitas, CA (US)

Correspondence Address: MORRISON & FOERSTER LLP, 755 PAGE MILL RD, PALO ALTO, CA, 94304-1018, US

(21) Appl. No.: 11/565,467

(22) Filed: Nov. 30, 2006

Related U.S. Application Data
(63) Continuation of application No. 10/814,555, filed on Mar. 30, 2004.
(60) Provisional application No. 60/459,470, filed on Mar. 30, 2003.
(60) Provisional application No. 60/478,127, filed on Jun. 11, 2003.

(51) Int. Cl. A61K 48/00; C07H 21/04

(52) U.S. Cl. 514/44; 536/23.1

(57) ABSTRACT

The invention provides methods for treating SLE including renal SLE and methods of reducing risk of renal flare in individuals with SLE, and methods of monitoring such treatment. One method of treating SLE including renal SLE and reducing risk of renal flare in an individual with SLE involves the administration of an effective amount of an agent for reducing the level of anti-dsDNA antibody (such as a dsDNA epitope as in the form of an epitope-presenting carrier or an epitope-presenting valency platform molecule like LJP 394) to the individual. The invention further provides a method of treating renal flare and reducing risk of renal flare in an individual with SLE involving the reduction of the level of circulating anti-dsDNA antibodies in the individual and maintaining sustained reduction of circulating anti-dsDNA antibodies, optionally through administration of a dsDNA epitope to the individual.

### CROSS-REFERENCES TO RELATED APPLICATIONS

This application is a continuation of U.S. patent application Ser. No. 10/814,555, filed Mar. 30, 2004, which claims the priority benefit of U.S. Provisional Application Nos. 60/459,470, filed Mar. 30, 2003, and 60/478,127, filed Jun. 11, 2003, all of which are incorporated in their entirety by reference.

### TECHNICAL FIELD

This invention relates to the field of antibody-mediated pathologies such as lupus. More particularly, the invention relates to methods of treating systemic lupus erythematosus (SLE) and methods of monitoring treatment of SLE in individuals.

### BACKGROUND OF THE INVENTION

Systemic lupus erythematosus (SLE) is characterized by multisystem organ involvement and variable disease course including flares and remissions. Renal disease is a primary cause of morbidity and mortality in SLE patients (Pistiner M, et al. (1991) Semin Arthritis Rheum 21:55-64, Hochberg M C, et al. (1985) Medicine 64:285-295, Dubois E L, et al. (1964) JAMA 190:104-11, Vitali C, et al. (1992) Clin Exp Rheumatol 10:527-39). In patients with SLE renal disease, high levels of anti-double stranded DNA antibodies (anti-dsDNA) correlate with active glomerulonephritis. A pathogenic role is suggested as these antibodies can be eluted from diseased glomeruli (Winfield J B, et al. (1977) J Clin Invest 59:90-6, Hahn, B. (1998) N Engl J Med 338:1359-68, Vlahakos D V, et al. (1992) Kidney Int 41:1690-700, Ehrenstein M R, et al. (1995) Kidney Int 48:705-11, Rothfield N F, et al. (1967) J Clin Invest 46:1785-94, Lefkowith J B, et al. (1996) J Clin Invest 98:1373-80). Significant increases in anti-dsDNA levels are associated with increased SLE disease activity; sustained reductions in antibody levels have been associated with improved treatment outcomes (Borg E J, et al. (1990) Arthritis Rheum, 33:634-43, Swaak A J G, et al. (1986) Ann Rheum Dis 45:359-66, Bootsma H, et al. (1995) Lancet 345:1595-9).

Although overall patient prognosis in SLE has improved, treatment regimens are not ideal and lupus nephritis continues to be associated with relatively poor overall survival as compared to individuals without renal involvement in lupus (Seleznick et al. (1991) Semin. Arthritis Rheum. 21:73-80). Acute episodes of nephritis are usually treated with high dose corticosteroids and/or immunosuppressive agents, typically cyclophosphamide, azathioprine, or recently mycophenolate mofetil. Poor tolerability, insufficient efficacy, and toxicity associated with these treatments limit their use, creating a need for alternative therapies (Klippel J H, et al. (1990) JAMA 263:1812-5, Ortmann R A, et al. (2000) Rheum Dis Clin North Am 26:363-75).

Synthetic double-stranded oligonucleotides (dsON) have been shown to cross-react with anti-dsDNA antibodies (U.S. Pat. No. 5,276,013). The use of dsON conjugated with non-immunogenic carriers, also referred to as platforms, has been proposed for a therapeutic approach for the treatment of SLE. For example, a tetrakis conjugate, LJP 249, composed of four dsON attached to a poly(ethylene glycol) valency platform was used to demonstrate tolerance in an immunized mouse model system (Jones et al. (1994) Bioconjugate Chem. 5:390-399).

LJP 394 (abetimus sodium), composed of 4 deoxynucleotide sequences bound to a triethylene glycol backbone, is a non-immunogenic, immunomodulatory agent, that selectively reduces anti-dsDNA titers in murine models of SLE and in patients with SLE (Plunkett et al. (1995) Lupus 4:S99, Coutts S M, et al. (1996) Lupus 5:158-9, Weisman M H (1997) J Rheumatol 24:314-38, Furie R A, et al. (2001) J Rheumatol 28:257-65). LJP 394 has been shown to induce antigen-specific B-cell tolerance in mice and rats, believed to occur by crosslinking anti-dsDNA antibodies on the surface of B cells resulting in anergy or apoptosis (Hartley S B, et al. (1993) Cell 72:325-35, Finkelman F D, et al. (1995) J Exp Med 181:515-25, Norvell A, et al. (1995) J Immunol 154:4404-13).

International Patent Application No. WO 01/41813 discloses methods of identifying lupus patients, including those with lupus nephritis, with high affinity anti-dsDNA antibodies and treatment of such patients with LJP 394. Other references discuss LJP394 in the context of a potential therapeutic agent for lupus. See Strand (2001) Lupus 10:216-221; Wallace (2001) Expert Opinion of Investigational Drugs 10:111-117; Furie et al. (2001) J. Rheumatol. 28:257-265.

Other literature describes methods which may be used in the treatment of SLE, including methods of reducing levels of circulating antibodies by inducing B cell tolerance, including, but not limited to, U.S. Pat. Nos. 5,276,013; 5,391,785; 5,786,512; 5,726,329; 5,552,391; 5,268,454; 5,606,047; 5,633,395; 5,162,515; U.S. Ser. No. 08/118,055 (U.S. Pat. No. 6,060,056); U.S. Ser. Nos. 60/088,656 and 60/103,088 (U.S. Ser. No. 09/328,199 and PCT App. No. PCT/US99/13194). See also U.S. Pat. Nos. 6,022,544; 6,340,460; 6,375,951; U.S. Pub. No. 2002/0187156; U.S. Pub. No. 2001/0010818 (U.S. Ser. No. 09/766,365) and WO 00/33887; U.S. Ser. No. 10/150,469 and WO 02/092011; U.S. Ser. No. 10/219,238.

A clinical trial of LJP 394 referred to as the 90-05 study, some accounts of which have been published as Linnik et al. (2000) Arth. Rheumat. 43(9 supplement):S241 (abstracts 1045 and 1046) and Alarcon-Segovia et al. (2000) Arth. Rheumat. 43(9 supplement):S272 (abstract 1231). See also, U.S. Ser. Nos. 09/457,875; 09/766,365; 10/115,806; 09/724,822; 10/748,541; U.S. Pub. No. 2003/0114405; WO 00/33887; WO 01/41813; PCT/US03/41840.

All references cited herein, including patents, patent applications and publications, are hereby incorporated by reference in their entirety.

### BRIEF SUMMARY OF THE INVENTION

In one aspect, the invention provides method of treating systemic lupus erythematosus (SLE) in an individual, comprising administering to the individual an effective amount of an agent which reduces anti-dsDNA antibody in the individual (such as, a dsDNA epitope which specifically binds to an anti-dsDNA antibody from the individual), wherein the administration of the agent results in a sustained reduction of anti-dsDNA antibody, and wherein the sustained reduction is at least about 10% below baseline in the individual (for example, a value of 100 at baseline would drop at least about 10% to about 90). In some embodiments, the sustained reduction is at least about 20% below baseline in the individual. In some embodiments, the sustained reduction is at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer. Ideally, treatment results in a sustained reduction for years, since SLE is a chronic disease. In some embodiments, the dsDNA epitope is the double-stranded polynucleotide 5′-GTGTGTGTGTGTGTGTGTGT-3′ (SEQ ID NO:1) in combination with its complementary strand, particularly the sequence 3′-CACACACACACACACACACA-5′ (SEQ ID NO:2), or one of the single-stranded polynucleotides 5′-GTGTGTGTGTGTGTGTGTGT-3′ (SEQ ID NO:1) or 3′-CACACACACACACACACACA-5′ (SEQ ID NO:2). The dsDNA epitope is optionally administered in the form of an epitope-presenting carrier. In other embodiments, the dsDNA epitope comprises, or, consists essentially of any of the above.

In another aspect, the invention provides a method of treating renal systemic lupus erythematosus (SLE) in an individual, comprising administering to the individual an effective amount of an agent which reduces anti-dsDNA antibody in the individual (such as, a dsDNA epitope which specifically binds to an anti-dsDNA antibody from the individual), wherein the administration of the agent results in a sustained reduction of anti-dsDNA antibody, and wherein the sustained reduction is at least about 10% below baseline in the individual (for example, a value of 100 at baseline would drop at least about 10% to about 90). In some embodiments, the sustained reduction is at least about 20% below baseline in the individual. In some embodiments, the sustained reduction is at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer. Ideally, treatment results in a sustained reduction for years, since SLE is a chronic disease. In some embodiments, the dsDNA epitope is the double-stranded polynucleotide 5′-GTGTGTGTGTGTGTGTGTGT-3′ (SEQ ID NO:1) in combination with its complementary strand, particularly the sequence 3′-CACACACACACACACACACA-5′ (SEQ ID NO:2), or one of the single-stranded polynucleotides 5′-GTGTGTGTGTGTGTGTGTGT-3′ (SEQ ID NO:1) or 3′-CACACACACACACACACACA-5′ (SEQ ID NO:2). The dsDNA epitope is optionally administered in the form of an epitope-presenting carrier. In other embodiments, the dsDNA epitope comprises, or, consists essentially of any of the above.

In one embodiment, the invention provides a method of treating systemic lupus erythematosus (SLE), including renal SLE, in an individual, comprising administering to the individual an effective amount of a conjugate comprising (a) a non-immunogenic valency platform molecule and (b) two or more double-stranded DNA (dsDNA) epitopes which specifically bind to an antibody from the individual which specifically binds to double-stranded DNA, wherein the administration of the conjugate results in sustained reduction of anti-dsDNA antibody, and wherein the sustained reduction is at least about 10% below baseline in the individual. In some embodiments, the sustained reduction is at least about 20% below baseline in the individual. In some embodiments, the sustained reduction is at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer. In one embodiment, the dsDNA epitope is optionally administered as the epitope-presenting valency platform molecule, such as the conjugate LJP 394 (Jones et al. (1995) J. Med Chem. 38:2138-2144).

In another aspect, the invention provides a method of treating systemic lupus erythematosus (SLE), including renal SLE, in an individual with systemic lupus erythematosus, comprising reducing the levels of circulating anti-dsDNA antibodies in the individual (for example, by administering an effective amount of an agent, such as a dsDNA epitope, which reduces anti-dsDNA antibody in the individual), and maintaining sustained reduction of the circulating anti-dsDNA antibodies, wherein the sustained reduction is at least about 10% below baseline in the individual, and wherein the sustained reduction of circulating anti-dsDNA antibodies in the individual results in reduction of incidence of renal flare. In some embodiments, the sustained reduction is at least about 20% below baseline in the individual. In some embodiments, the sustained reduction is at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer.

In one embodiment, the levels of circulating anti-dsDNA antibodies in the individual are reduced by administration of an effective amount of a dsDNA epitope, such as the double-stranded polynucleotide 5′-GTGTGTGTGTGTGTGTGTGT-3′ (SEQ ID NO:1) and its complementary strand, particularly the sequence 3′-CACACACACACACACACACA-5′ (SEQ ID NO:2) or one of the single-stranded polynucleotides 5′-GTGTGTGTGTGTGTGTGTGT-3′ (SEQ ID NO:1) or 3′-CACACACACACACACACACA-5′ (SEQ ID NO:2) to the individual. In another embodiment, the levels of circulating anti-dsDNA antibodies in the individual are reduced by administering to the individual an effective amount of epitope-presenting valency platform molecule, such as the conjugate LJP 394.

In another aspect, the invention provides a method of reducing risk of renal flare in an individual with systemic lupus erythematosus, comprising reducing the levels of circulating anti-dsDNA antibodies in the individual (for example, by administering an effective amount of an agent, such as a dsDNA epitope, which reduces anti-dsDNA antibody in the individual), and maintaining sustained reduction of the circulating anti-dsDNA antibodies, wherein the sustained reduction is at least about 10% below baseline in the individual. In some embodiments, the sustained reduction of circulating anti-dsDNA antibodies in the individual results in reduction of incidence of renal flare. In some embodiments, the sustained reduction is at least about 20% below baseline in the individual. In some embodiments, the sustained reduction is at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer.

In another aspect, the invention provides a method of reducing risk of Major SLE flare in an individual with systemic lupus erythematosus, comprising reducing the levels of circulating anti-dsDNA antibodies in the individual (for example, by administering an effective amount of an agent, such as a dsDNA epitope, which reduces anti-dsDNA antibody in the individual), and maintaining sustained reduction of the circulating anti-dsDNA antibodies, wherein the sustained reduction is at least about 10% below baseline in the individual. In some embodiments, the sustained reduction is at least about 20% below baseline in the individual. In some embodiments, the sustained reduction is at least about 30% below baseline in the individual. In some embodiments, the sustained reduction of circulating anti-dsDNA antibodies in the individual results in reduction of incidence of Major SLE flare. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer.

In another aspect, the invention provides a method of reducing incidence of Major SLE flare in an individual with systemic lupus erythematosus, comprising administering to the individual an effective amount of an agent which reduces the levels of circulating anti-dsDNA antibodies in the individual, wherein the administration of the agent results in a sustained reduction of the circulating anti-dsDNA antibodies, and wherein the sustained reduction is at least about 10% below baseline in the individual. In some embodiments, the sustained reduction is at least about 20% below baseline in the individual. In some embodiments, the sustained reduction is at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer.

In another aspect, the invention provides a method of reducing risk of hospitalization in an individual with systemic lupus erythematosus (SLE), comprising reducing the levels of circulating anti-dsDNA antibodies in the individual (for example, by administering an effective amount of an agent, such as a dsDNA epitope, which reduces anti-dsDNA antibody in the individual), and maintaining sustained reduction of the circulating anti-dsDNA antibodies, wherein the sustained reduction is at least about 10% below baseline in the individual. In another aspect, the invention also provides a method of reducing risk of SLE related hospitalization in an individual with SLE, comprising reducing the levels of circulating anti-dsDNA antibodies in the individual (for example, by administering an effective amount of an agent, such as a dsDNA epitope, which reduces anti-dsDNA antibody in the individual), and maintaining sustained reduction of the circulating anti-dsDNA antibodies, wherein the sustained reduction is at least about 10% below baseline in the individual. In some embodiments, the sustained reduction is at least about 20% below baseline in the individual. In some embodiments, the sustained reduction is at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer.

In one embodiment, the levels of circulating anti-dsDNA antibodies in the individual are reduced and the sustained reduction is maintained by administration of an effective amount of a dsDNA epitope, such as the double-stranded polynucleotide 5′-GTGTGTGTGTGTGTGTGTGT-3′ (SEQ ID NO:1) and its complementary strand, particularly the sequence 3′-CACACACACACACACACACA-5′ (SEQ ID NO:2) or one of the single-stranded polynucleotides 5′-GTGTGTGTGTGTGTGTGTGT-3′ (SEQ ID NO:1) or 3′-CACACACACACACACACACA-5′ (SEQ ID NO:2) to the individual. In another embodiment, the levels of circulating anti-dsDNA antibodies in the individual are reduced by administering to the individual an effective amount of epitope-presenting valency platform molecule, such as the conjugate LJP 394.

In another aspect, the invention provides methods of monitoring a treatment for SLE, including renal SLE, in an individual, said method comprising measuring level of anti-dsDNA antibody in the individual, wherein a sustained reduction of circulating anti-dsDNA antibody of at least about 10% below baseline indicates effectiveness of the treatment for renal flare. In some embodiments, the sustained reduction is at least about 20% below baseline in the individual. In some embodiments, the sustained reduction is at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer. In some embodiments, said treatment comprises administration of a conjugate comprising (a) a non-immunogenic valency platform molecule and (b) two or more dsDNA epitopes, preferably polynucleotides, which specifically bind to an antibody from the individual which specifically binds to double stranded DNA. In one embodiment, the conjugate is LJP 394.

In another aspect, the invention provides methods of indicating likelihood of success of treatment for SLE including renal SLE (or in other embodiments, likelihood of success of (a) reducing risk of renal flare, (b) reducing risk of Major SLE flare), said method comprising measuring level of circulating anti-dsDNA antibody in the individual, wherein a sustained reduction of anti-dsDNA antibody of at least about 10% below baseline indicates likelihood of success of the treatment for renal flare (or in other embodiments, likelihood of success of (a) reducing risk of renal flare, (b) reducing risk of Major SLE flare). In some embodiments, the sustained reduction is at least about 20% below baseline in the individual. In some embodiments, the sustained reduction is at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer. In some embodiments, said treatment comprises administration of a conjugate comprising (a) a non-immunogenic valency platform molecule and (b) two or more dsDNA epitopes, preferably polynucleotides, which specifically bind to an antibody from the individual which specifically binds to double stranded DNA. In one embodiment, the conjugate is LJP 394.

In the aspects and the embodiments described above, an individual of particular interest is a human.

As described herein, the sustained reduction may be effected in a number of ways, including administration of an agent (such as a dsDNA epitope) which effects the sustained reduction.

As described herein, the sustained reduction may be any extent (in terms of percentage) as described herein, and/or for any duration as described herein.

In another aspect, the invention provides methods of assessing increased risk of renal flare comprising determining whether the individual has a sustained increases in anti-dsDNA antibodies (by comparing measurements as described herein). The levels of anti-dsDNA antibodies as compared to baseline are as described herein. In some embodiments, the sustained increases in anti-dsDNA antibodies are at least about 10% above baseline for greater than or equal to about ⅔ of all observed values.

### BRIEF DESCRIPTION OF THE DRAWINGS

FIG. 1 is a graph depicting median percentage change of antibodies to dsDNA in the sustained reduction group and in the other group (with placebo and LJP 394 treated shown separately) in Phase III trial.

FIG. 2 is a graph depicting median percentage change of antibodies to dsDNA in the sustained reduction group and in the other group (with placebo and LJP 394 treated shown separately) in Phase II/III trial.

FIG. 3 is a graph depicting median percentage change of antibodies to dsDNA in the sustained reduction group and in the other group (including both placebo and LJP 394 treated) in Phase III trial.

FIG. 4 is a graph depicting median percentage change of antibodies to dsDNA in the sustained reduction group and in the other group (including both placebo and LJP 394 treated) in Phase II/III trial.

### DETAILED DESCRIPTION OF THE INVENTION

We have observed a correlation between level of circulating anti-dsDNA antibody and incidence of renal flare and Major SLE flare in patients with systemic lupus erythematosus (SLE) including patients with renal SLE. Based on our evaluation of clinical data, we have observed the following: (a) patients with sustained reductions in circulating anti-dsDNA antibody of at least about 10% below baseline are less likely to have renal flares when compared to patients without sustained reductions in circulating anti-dsDNA antibody; (b) increase in circulating anti-dsDNA antibody correlates with increase of incidence of renal flare; (c) sustained rises of at least about 10% above baseline correlate with increased risk of renal flare; (d) decrease in circulating anti-dsDNA correlates with increase in complement (C3) level; (e) anti-dsDNA antibodies are pathogenic; (f) patients with sustained reductions in anti-dsDNA antibodies have a lower risk of Major SLE flare when compared with patients that did not have sustained reductions; (g) patients with sustained reductions in anti-dsDNA antibodies have fewer hospitalization including SLE related hospitalization when compared with patients that did not have sustained reductions.

Accordingly, the invention provides methods of treatment for SLE, including renal SLE, and methods of reduction of risk of renal flare and risk of Major SLE flare by reducing (e.g., by administering an agent that reduces) anti-dsDNA in an individual and maintaining sustained reduction of anti-dsDNA antibody of at least about 10% below baseline. For purpose of this invention, sustained reduction is defined as having at least about 10% reduction below baseline in anti-dsDNA antibody for at least majority of the times that the agent is administered. In some embodiments, the anti-dsDNA antibody levels are at least about 10% reduction below baseline for greater than or equal to about ⅔ of all observed values prior to HDCC or last (most recent) dose of a drug (for example, LJP394) used for treatment, or about ⅔ of the values measured during treatment. The term “baseline” refers to the mean of the last two determinations of the circulating anti-dsDNA antibody level in an individual prior to or upon initial administration of the drug. A baseline may also be established by a measurement of the level of anti-dsDNA antibody prior to or upon initial administration of the drug. A baseline may also be determined as above with reference to prior to or initiation of any treatment (such as administration of a drug (agent) or procedure). The invention also provides methods of monitoring the effectiveness of an SLE treatment and methods of indicating likelihood of success of treatment for renal flare by measuring the level of anti-dsDNA antibody.

Reduction below baseline can be achieved by a system of monitoring whereby agent is administered but may be reduced or even eliminated when individual shows sustained reduction.

In some embodiments, anti-dsDNA antibody levels are measured weekly. In other embodiments, anti-dsDNA antibody levels are measured monthly. If requisite sustained reduction appears to be established, less frequent (and/or variable) measurements may be made.

It is understood that a percentage reduction required for sustained reduction may vary over time; for example, an individual may have at least about a 20% reduction (for a given period of time), but may have or require at least 10% reduction.

In some embodiments of the invention, the sustained reduction is at least about 20% below baseline in anti-dsDNA antibody. In some embodiments, the sustained reduction is at least about 25% below baseline in the individual. In some embodiments, the sustained reduction is at least about 30% below baseline in the individual. In some embodiments, the sustained reduction is at least about 35% below baseline in the individual. In some embodiments, the sustained reduction is at least about 40% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer (as SLE is a chronic disease that can last a lifetime).

The invention also provides methods of assessing increased risk of renal flare by determining whether the individual has a sustained increases in anti-dsDNA antibodies.

I. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991) and Short Protocols in Molecular Biology (Wiley and Sons, 1999). Other useful references include Harrison's Principles of Internal Medicine (McGraw Hill; J. Isseleacher et al., eds.) and Dubois' Lupus Erythematosus (5th ed.; D. J. Wallace and B. H. Hahn, eds.; Williams & Wilkins, 1997).

II. Definitions

A “population” is a group of individuals with lupus and/or renal lupus.

“SLE flares” are used herein to refer to flares (i.e. acute clinical events) which occur in patients with SLE. The SLE flares may be in various major organs, including but not limited to, kidney, brain, lung, heart, liver, and skin. If the activity is in the kidneys, then the SLE flare is referred to as a “renal flare”. “Renal flares” can be identified by evaluating factors including, but not limited to, proteinuria levels, hematuria levels, and serum creatinine levels.

A “Major SLE flare” is used herein to refer to the occurrence of any one or more of the following due to manifestations of active SLE: treatment with HDCC or initiation or increase in treatment with other immunosuppressive agents, including azathioprine, mycophenolate mofetil, methotrexate, cyclosporin and leflunomide; or hospitalization or death.

“Reducing incidence” of renal flares or Major SLE flares in an individual with SLE means any of reducing severity (which can include reducing need for and/or amount of (e.g., exposure to) other drugs generally used for this conditions, including, for example, high dose corticosteroid and/or cyclophosphamide), duration, and/or frequency (including, for example, delaying or increasing time to renal flare as compared to not receiving treatment) of renal flare(s) in an individual. As is understood by those skilled in the art, individuals may vary in terms of their response to treatment, and, as such, for example, a “method of reducing incidence of renal flares in an individual” reflects administering the epitope(s) described herein based on a reasonable expectation that such administration may likely cause such a reduction in incidence in that particular individual.

“High dose corticosteroid and/or cyclophosphamide” or “HDCC” as used herein refers to intervention with an increased dosage of corticosteroid alone or with cyclophosphamide. High dose generally refers to corticosteroids. Such intervention generally occurs upon a flare, or acute episode. Generally, for example, the increased dosage is at least a 15 mg/day and can be greater than 20 mg/day. HDCC may be administered using standard clinical protocols. A clinician may monitor a patient and determine when HDCC treatment is needed by evaluating factors including, but not limited to, proteinuria levels, hematuria levels, and serum creatinine levels. In general, patients who experience renal flares are given HDCC treatment, although this treatment is used for other aspects of lupus.

As used herein, the singular form “a”, “an”, and “the” includes plural references unless indicated otherwise. For example, “an” antibody includes one or more antibodies.

An “epitope” is a term well-understood in the art and means any chemical moiety that exhibits specific binding to an antibody. An “epitope” can also comprise an antigen, which is a moiety or molecule that contains an epitope, and, as such, also specifically binds to antibody.

A “double-stranded DNA epitope” or “dsDNA epitope” (used interchangeably herein) is any chemical moiety which exhibits specific binding to an anti-double-stranded DNA antibody and, as such, includes molecules which comprise such epitope(s). Further discussion of double-stranded DNA epitopes suitable for use in the methods of the invention are described below. The term “dsDNA epitope” also includes mimetics of double-stranded DNA itself, which are described below. Examples of analogs or mimetics of double-stranded DNA that are encompassed by the term “dsDNA epitope” include, but are not limited to, (a) single-stranded DNA polynucleotides that preferentially bind anti-dsDNA antibodies and (b) the pentapeptide Asp/Glu-Trp-Asp/Glu-Tyr-Ser/Gly (DeGiorgio et al. (2001) Nature Medicine 7:1189-1193; Putterman and Diamond (1998) J. Exp. Med. 188:29-38; Gaynor et al. (1997) Proc. Natl. Acad. Sci. USA 94:1955-1960).

An epitope that “specifically binds” or “preferentially binds” (used interchangeably herein) to an antibody or a polypeptide is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a double-stranded DNA (dsDNA) epitope is an antibody that binds the dsDNA epitope with greater affinity, avidity, more readily, and/or with greater duration than it binds to non-ds DNA epitopes. It is also understood by reading this definition that, for example, an antibody (or moiety or epitope) that specifically binds to a first target may or may not specifically or preferentially bind to a second target. As such, “specific binding”, “specifically binding”, “preferentially binding”, or “preferential binding” does not necessarily require (although it can include) exclusive binding. For instance, a cross-reacting antibody that specifically binds or preferentially binds a dsDNA epitope may also specifically bind or preferentially bind the N-methyl-D-aspartate (NMDA) receptors NR2a or NR2b (DeGiorgio, et al. (2001) Nature Medicine 7:1189-1193; Putterman and Diamond, (1998) J. Exp. Med. 188:29-38; Gaynor et al., Proc. Natl. Acad. Sci. USA 94:1955-1960.). Also, an antibody that specifically binds or preferentially binds a dsDNA epitope may also specifically bind or preferentially bind a single-stranded DNA molecule.

An “anti-double-stranded DNA antibody” or “anti-dsDNA antibody” or “double-stranded DNA antibody” or “antibodies to dsDNA”, used interchangeably herein, is any antibody which specifically binds to double-stranded DNA (dsDNA). This term is used to generally refer to SLE-associated antibodies, which are antibodies whose production occurs during an SLE disease state and/or whose production is undesirable in a patient with SLE. An “anti-ds DNA antibody” can also specifically bind to a single-stranded DNA, and as such, this term includes antibodies which cross-react with single-stranded DNA, although such cross-reactivity is not required. The “ds” terminology is used in accordance with the traditional nomenclature in this field. As such, based on this definition, these antibodies could also be termed “anti-DNA” antibodies. Any antibody includes an antibody of any class, such as IgG, IgA, or IgM, and the antibody need not be of any particular class. As clearly indicated in the definition of “antibody” provided herein, an “anti-double-stranded DNA antibody” encompasses any fragment(s) that exhibits this requisite functional (i.e., specific binding to dsDNA) property, such as fragments that contain the variable region, such as Fab fragments. As discussed below, it is understood that specific binding to any anti-double-stranded DNA antibody (or functional fragment) is sufficient. Optionally, an anti-dsDNA antibody may cross-react with mimetics or analogs of the dsDNA epitope. For instance, an anti-dsDNA antibody may cross-react with the pentapeptide sequence Asp/Glu-Trp-Asp/Glu-Tyr-Ser/Gly, such as that found in the N-methyl-D-aspartate (NMDA) receptor NR2a and N-methyl-D-aspartate (NMDA) receptor NR2b (DeGiorgio, et al. (2001)).

The terms “circulating anti-double-stranded DNA antibody”, “circulating anti-dsDNA antibody”, and “circulating SLE-associated antibody”, as used herein, intends an anti-double-stranded DNA antibody which is not bound to a double-stranded DNA epitope on and/or in a biological sample, i.e., free antibody.

For purposes of this invention, “reducing” and/or “removing” SLE-associated circulating antibodies means that the level of free, or unbound, circulating SLE-associated antibodies has been reduced. Circulating SLE-associated antibodies are optionally reduced or removed by the binding of circulating SLE-associated antibodies to an administered moiety or by the induction of tolerance, including the induction of B cell anergy. In some embodiments, by binding of epitope to an antibody, the antibody is prevented from being an effector molecule, i.e., binding other targets, and is thus “reduced.” In some embodiments, “reducing” circulating antibodies includes clearance of antibody, e.g., physical removal from circulation. One way clearance is effected is clearance of a complex comprising an epitope carrier, such as an epitope-presenting valency platform molecule, and antibody by reticuloendothelial system.

An “antibody” (interchangeably used in plural form) is an immunoglobulin molecule capable of specific binding to a target, such as a carbohydrate, polynucleotide or polypeptide, through at least one antigen recognition site, located in the variable region of the immunoglobulin molecule. As used herein, the term encompasses not only intact antibodies, but also fragments thereof (such as Fab, Fab′, F(ab′)₂, Fv), single chain (ScFv), mutants thereof, fusion proteins comprising an antibody portion, humanized antibodies, and any other modified configuration of the immunoglobulin molecule that comprises an antigen recognition site of the required specificity.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. It is understood that the double stranded polynucleotide sequences described herein also include the modifications described herein. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P-NH2) or a mixed phosphoramidate-phosphodiester oligomer. A phosphorothioate linkage can be used in place of a phosphodiester linkage. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. For purposes of this invention, a polynucleotide is generally an isolated polynucleotide of less than about 1 kb, preferably less than about 500 base pairs (bp), preferably less than about 250 bp, preferably less than about 100 bp, preferably less than about 50 bp. However, it is understood that a polynucleotide of any size or configuration could be used as long as it exhibits the requisite binding to anti dsDNA antibody from an individual. It is further understood that a different polynucleotide (for example, in terms of size and/or sequence) other than the one that is to be, was, or will be used in treatment, as long as both polynucleotides exhibit equivalent (or convertible) binding affinities to anti-dsDNA antibodies from an individual. In other words, non-identical polynucleotides may be employed with respect to affinity determination and treatment.

Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

As used herein, an “analog” or “mimetic” of an epitope means a biological or chemical compound which specifically binds to an antibody to which the epitope specifically binds. As such, a “double-stranded DNA epitope” includes mimetics of naturally-occurring double-stranded DNA. An “analog” or “mimetic” of a dsDNA epitope shares an epitope, or binding specificity, with double-stranded DNA. An analog or mimetic may be any chemical substance which exhibits the requisite binding properties, and thus may be, for example, a simple or complex organic or inorganic molecule; a polypeptide; a polynucleotide; a carbohydrate; a lipid; a lipopolysaccharide; a lipoprotein, or any combination of the above, including, but not limited to, a polynucleotide-containing polypeptide; a glycosylated polypeptide; and a glycolipid. The term “analog” encompasses the term “mimotope”, which is a term well known in the art.

An “individual” is a vertebrate, preferably a mammal, more preferably a human. Mammals include, but are not limited to, farm animals, sport animals, pets, primates, mice and rats.

“Inducing tolerance” or “inducing immunotolerance” means a reduction and/or stabilization of the extent of an immune response to an immunogen, and, as such, means immune unresponsiveness (or at least a reduction in the extent of an immune response) at the organismal level and unresponsiveness (e.g., anergy) and/or apoptosis at the cellular level. An “immune response” may be humoral and/or cellular, and may be measured using standard assays known in the art. For purposes of this invention, the immune response is generally reflected by the presence of, and/or the levels of, anti-double-stranded DNA antibodies. Quantitatively the reduction (as measured by reduction in antibody production and/or levels) is at least about 15%, preferably at least about 25%, more preferably at least about 50%, more preferably at least about 75%, more preferably at least about 90%, even more preferably at least about 95%, and most preferably 100%. It is understood that the tolerance is antigen-specific, and applies for purposes of the invention to those individuals having anti-double-stranded DNA antibodies. “Inducing tolerance” also includes slowing and/or delaying the rate of increase of antibody level.

As used herein, the term “B cell anergy” intends unresponsiveness of those B cells requiring T cell help to produce and secrete antibody and includes, without limitation, clonal deletion of immature and/or mature B cells and/or the inability of B cells to produce antibody. “Unresponsiveness” means a therapeutically effective reduction in the humoral response to an immunogen. Quantitatively the reduction (as measured by reduction in antibody production) is at least 50%, preferably at least 75% and most preferably 100%.

An “effective amount” (when used in the lupus context, or in the antibody-mediated pathology context) is an amount sufficient to effect beneficial or desired results including clinical results. An effective amount can be administered in one or more administrations. For purposes of this invention, an effective amount of an agent, an epitope, epitope-presenting carrier, or an epitope-presenting valency platform molecule described herein (or a composition comprising the same), is an amount sufficient to maintain sustained reduction of circulating anti-dsDNA antibody of at least about 10% below baseline, optionally by inducing tolerance, particularly with respect to anti-double-stranded DNA antibodies.

An “isolated” or “purified” polypeptide or polynucleotide is one that is substantially free of the materials with which it is associated in nature. By substantially free is meant at least 50%, preferably at least 70%, more preferably at least 80%, even more preferably at least 90% free of the materials with which it is associated in nature.

A “carrier”, as used herein, is a molecule which contains at least one attachment site for an epitope. One example of a carrier is a valency platform molecule.

As used herein “valency platform molecule” means a nonimmunogenic molecule containing sites which allow the attachment of a discrete number of epitopes and/or mimetic(s) of epitopes. A “valency” of a conjugate or valency platform molecule indicates the number of attachment sites per molecule for a double-stranded DNA epitope(s). Alternatively, the valency of a conjugate is the ratio (whether absolute or average) of double-stranded DNA epitope to valency platform molecule.

“Nonimmunogenic”, when used to describe the valency platform molecule, means that the valency platform molecule fails to elicit an immune response (i.e., T cell and/or B cell response), and/or fails to elicit a sufficient immune response, when it is administered by itself to an individual. The degree of acceptable immune response depends on the context in which the valency platform molecule is used, and may be empirically determined.

An epitope which is “conjugated” to a carrier or a valency platform molecule is one that is attached to the carrier or valency platform molecule by covalent and/or non-covalent interactions.

An “epitope-presenting carrier” is a carrier which contains at least one attached, or bound, epitope which is specifically bound by an antibody of interest (such as an SLE-associated antibody). Optionally, a carrier contains attached, or bound, epitopes, at least two of which are able to bind to an antibody of interest.

An “epitope-presenting valency platform molecule” is a valency platform molecule which contains attached, or bound, epitopes, at least some of which (at least two of which) are able to bind an antibody of interest.

“In conjunction with” refers to administration of one treatment modality in addition to another treatment modality, such as administration of a dsDNA epitope described herein, in addition to administration of a psychiatric medication, such as an anti-depressant, to the same individual. As such, “in conjunction with” refers to administration of one treatment modality before, during or after delivery of the other treatment modality to the individual.

An individual having “renal disease” or “renal impairment” (which, in some embodiments depending on clinical indicia, includes significant impaired renal function or significant renal impairment) is an individual exhibiting one or more clinical signs of renal dysfunction, as described herein. Clinical signs of renal dysfunction include anuria, oliguria, elevated blood urea nitrogen (BUN), elevated serum creatinine, clinically significant proteinuria, hematuria, reduced creatinine clearance, and other clinical indications of renal dysfunction known in the art. As described herein, generally, an individual displays renal disease if any one of more of these clinical indicia are at least above the upper limit of “normal” range, as defined in the clinical arts. In some embodiments, renal disease is indicated if the value exceeds the upper limit of normal by about any of the following percentages: 10, 20, 25, 30, 50, 60, 75, 100, 125, 150, 200, 250, 275, 300, 350, 400, 450, 500. As is known in the art, with respect to at least one indicia of kidney function, such as serum creatinine, an individual can have at least about 2, 3, 5, or 10 fold or greater values compared with the upper limit of normal. In some embodiments, an individual having a significant renal impairment can have more than 1.5 mg/dL serum creatinine. Generally, an individual is determined to have, or in fact has, renal disease at the onset (before the individual receives the first administration), or shortly after the onset (within about 4 weeks, preferably within about 2 weeks, preferably within about 1 week, preferably within about 5 days, preferably within about 2 days, preferably within about 1 day) upon receiving the first administration), of the therapeutic methods described herein.

When renal disease (including significant impaired renal function) “is used as a basis” for administration of the treatment methods described herein, or selection for the treatment methods described herein, renal function is measured before and/or during treatment, and the values obtained are used by a clinician in assessing probable or likely suitability of an individual to receive treatment(s). As would be well understood by one in the art, measurement of renal function in a clinical setting is a clear indication that this parameter was used as a basis for initiating, continuing, adjusting and/or ceasing administration of the treatments described herein.

“Affinity” of an antibody from an individual for an epitope to be used, or used, in treatment(s) described herein is a term well understood in the art and means the extent, or strength, of binding of antibody to epitope. Affinity may be measured and/or expressed in a number of ways known in the art, including, but not limited to, equilibrium dissociation constant (K_{D} or K_{d}), apparent equilibrium dissociation constant (K_{D}′ or K_{d}′), and IC₅₀ (amount needed to effect 50% inhibition in a competition assay; used interchangeably herein with “I₅₀”). It is understood that, for purposes of this invention, an affinity is an average affinity for a given population of antibodies which bind to an epitope. Values of K_{D}′ reported herein in terms of mg IgG per mL or mg/mL indicate mg Ig per mL of serum, although plasma can be used.

When antibody affinity “is used as a basis” for administration of the treatment methods described herein, or selection for the treatment methods described herein, antibody affinity is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. As would be well understood by one in the art, measurement of antibody affinity in a clinical setting is a clear indication that this parameter was used as a basis for initiating, continuing, adjusting and/or ceasing administration of the treatments described herein.

An antibody affinity measured “before or upon initiation of treatment” or an “initial affinity” is antibody affinity measured in an individual before the individual receives the first administration of a treatment modality described herein and/or within at least about 4 weeks, preferably within at least about 2 weeks, preferably within at least about 1 week, preferably within at least about 5 days, preferably within at least about 3 days, preferably within at least about 2 days, preferably within at least about 1 day upon receiving the first administration of a treatment modality described herein.

An “equivalent” or “functional equivalent” of K_{D}′ or a numerical value for K_{D}′ is a parameter or value for a parameter which also reflects affinity. For example, an equivalent of K_{D}′ is IC₅₀. As another example, an equivalent value of K_{D}′ of 0.5 could be an IC₅₀ of 200, if they reflect the same, or about the same, affinity. Determining such equivalents is well within the skill of the art and such equivalents and their determination are encompassed by this invention. Generally, reference to K_{D}′ includes reference to functional equivalents of K_{D}′.

“Receiving treatment” includes initial treatment and/or continuing treatment. As used herein, “treatment” is an approach for obtaining beneficial or desired results, preferably including clinical results. For purposes of this invention, beneficial or desired clinical results include, but are not limited to, one or more of the following: alleviation of one or more symptoms, diminishment of extent of disease, stabilized (i.e., not worsening) state of disease, preventing spread of disease, preventing occurrence or recurrence of disease, decreasing, delaying or preventing the occurrence of renal “flares,” amelioration of the disease state, remission (whether partial or total), reduction of incidence of disease and/or symptoms, stabilizing (i.e., not worsening) of renal function or improvement of renal function. During lupus nephritis, which is a chronic inflammatory kidney disease, “flares” may occur. “Flares” refer to an increase in activity, generally inflammatory activity. If the activity is in the kidneys, then the flare is referred to as a “renal flare”. “Renal flares” can be identified by evaluating factors including, but not limited to, proteinuria levels, hematuria levels, and serum creatinine levels. The “treatment” of lupus nephritis may be administered when no symptoms of lupus nephritis are present, and such treatment (as the definition of “treatment” indicates) reduces the incidence of flares. Also encompassed by “treatment” is a reduction of pathological consequences of any aspect of lupus renal disease (including nephritis).

An individual who “may be suitable”, which includes an individual who is “suitable” for treatment(s) described herein, is an individual who is more likely than not to benefit from administration of said treatments. Conversely, an individual who “may not be suitable” or “may be unsuitable”, which includes an individual who is “unsuitable” for treatment(s) described herein, is an individual who is more likely than not to fail to benefit from administration of said treatments.

III. Methods of Treatment

In one aspect, the invention provides a method of treating systemic lupus erythematosus (SLE), including renal SLE, in an individual, comprising administering to the individual an effective amount of an agent which decreases anti-dsDNA antibody in the individual (such as a dsDNA epitope which specifically binds to an anti-dsDNA antibody from the individual (generally, an antibody which specifically binds to double-stranded DNA, although as is known in the art, and described herein, such antibodies may also bind single-stranded DNA and/or mimetics or analogs of dsDNA such as the pentapeptide sequence Asp/Glu-Trp-Asp/Glu-Tyr-Ser/Gly)), wherein the administration of the agent results in sustained reduction of anti-dsDNA antibody of at least about 10% below baseline in the individual. In some embodiments, the sustained reduction is for at least about 20% below baseline in the individual. In some embodiments, the sustained reduction is for at least about 30% below baseline in the individual. In one embodiment, the dsDNA epitope is administered to the individual in the form of an epitope-presenting carrier. Optionally, the epitope-presenting carrier is an epitope-presenting valency platform molecule comprising (a) a non-immunogenic valency platform molecule and (b) two or more double-stranded DNA (dsDNA) epitopes which specifically bind to an antibody from the individual which specifically binds to double-stranded DNA, wherein the administration of the conjugate results in sustained reduction of anti-dsDNA antibody of at least about 10% below baseline in the individual. Exemplary embodiments of an epitope-presenting valency platform molecule are described herein. In some embodiments, the sustained reduction is for at least about 20% below baseline in the individual. In some embodiments, the sustained reduction is for at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer.

In another aspect, the invention provides a method of treating SLE, including renal SLE, an individual, comprising reducing the levels of circulating anti-dsDNA antibodies in the individual, and maintaining a sustained reduction of the anti-dsDNA antibodies in the individual of at least about 10% below baseline, wherein sustained reduction of the levels of the circulating anti-dsDNA antibodies in the individual results in reduction of incidence of renal flare. In one embodiment, the anti-dsDNA antibodies in the individual are antibodies that specifically bind double-stranded DNA and single-stranded DNA. In one embodiment, the anti-dsDNA circulating antibodies bind either strand or both strands of the double-stranded polynucleotide comprising, consisting of, or consisting essentially of a strand having the sequence 5′-GTGTGTGTGTGTGTGTGTGT-3′ (SEQ ID NO:1) and the complementary strand 3′-CACACACACACACACACACA-5′ (SEQ ID NO:2). Optionally, the anti-dsDNA antibodies bind one of the single-stranded polynucleotides 5′-GTGTGTGTGTGTGTGTGTGT-3′ (SEQ ID NO:1) or 3′-CACACACACACACACACACA-5′ (SEQ ID NO:2). In another embodiment, the anti-dsDNA antibodies specifically bind the pentapeptide sequence Asp/Glu-Trp-Asp/Glu-Tyr-Ser/Gly. In some embodiments, the sustained reduction is for at least about 20% below baseline in the individual. In some embodiments, the sustained reduction is for at least about 30% below baseline in the individual. In some embodiments, the sustained reduction is for at least about one month. In some embodiments, the sustained reduction is for at least about two months. In some embodiments, the sustained reduction is for at least about three months. In some embodiments, the sustained reduction is for at least about four months. In some embodiments, the sustained reduction is for at least about five months. In some embodiments, the sustained reduction is for at least about six months. In some embodiments, the sustained reduction is for at least about one year. In some embodiments, the sustained reduction is for at least about two years or longer.

In one embodiment, the anti-dsDNA antibodies are optionally reduced by binding circulating anti-dsDNA antibodies and/or by inducing tolerance, including by inducing B cell anergy.

Reference to circulating anti-dsDNA antibodies are exemplary and also apply to anti-dsDNA antibodies in general.

In some embodiments, the levels of circulating anti-dsDNA antibodies are reduced by at least about any one of the following amounts: 10%, 15%, 20%, 25%, 30%, 40%, 50%, 60%, 70%, 80%, 90%, or 95%. In some embodiments, the levels of circulating anti-dsDNA antibodies are reduced by at least about 20%, 25%, 30%, or 40%. In some alternative embodiments, the levels of circulating anti-dsDNA antibodies are reduced from about 10% to about 95%, from about 10% to about 70%, from about 15% to about 40%, or from about 20% to about 35%. It is understood that, for purposes of this invention, total reduction (i.e., 100%) need not be effective in order for these methods to be efficacious. Methods of measuring antibody titer, either by binding or neutralizing assays, are well known in the art.

In some embodiments, the level of circulating anti-dsDNA antibodies are reduced and maintained at a sustained reduction of at least about 10% below baseline level. Sustained reduction is a reduction of at least about 10% reduction below baseline in anti-dsDNA antibody for at least majority of the times that the agent is administered. In some embodiments, the anti-dsDNA antibody levels are at least about 10% reduction below baseline for greater than or equal to about ⅔ of all observed values measured prior to HDCC or last (most recent) dose of a drug (for example, LJP 394) used for treatment, or about ⅔ of the values measured during treatment. Baseline anti-dsDNA antibody levels are calculated as the mean of the last two determinations prior to or upon initial administration of the drug. A baseline may also be established by a measurement of the level of anti-dsDNA antibody prior to or upon initial administration of the drug. In some embodiments, the sustained reduction is at least about 20% below baseline. In some embodiments, the sustained reduction is at least about 25% below baseline. In some embodiments, the sustained reduction is at least about 30% below baseline. In some embodiments, the sustained reduction is for at least about one month, at least about two months, at least about three months, at least about four months, at least about 16 weeks, at least about five months, at least about six months (at least about 24 weeks), at least about 48 weeks, at least about 1 year, or at least about two years or longer.

Methods of measuring antibody titer, either by binding or neutralizing assays, are well known in the art. Three assays are currently used in the diagnosis of SLE by measuring anti-dsDNA antibodies; these are the Farr, Crithidia, and ELISA assays. The Farr assay is considered by clinicians to be the most specific and sensitive of the three and the most useful in the prediction of flares in disease activity, especially renal flares (Smeenk et al., Rheumatol. Int. 11:101-7 (1991); ter Borg et al., Arthritis Rheum. 33:634-43 (1990)).

In some embodiments, the anti-dsDNA antibodies may be measured weekly. In other embodiments, the antibodies are measured every two weeks. In other embodiments, the antibodies are measured monthly. If requisite sustained reduction appears to be established, less frequent (or variable) measurements may be made.

In one embodiment, the levels of circulating anti-dsDNA antibodies are reduced by administration of a dsDNA epitope to the individual. Optionally, the dsDNA epitope is administered to the individual in the form of an epitope-presenting carrier. For instance, the published patent application, Taylor et al., United States Patent Application No. 20020103343 describes constructs comprising at least one monoclonal antibody specific for binding to complement receptor (CR1) site on primate erythrocytes, where the antibody is crosslinked to an antigen specific for a target pathogenic autoantibody, such as an anti-dsDNA antibody.

In one embodiment of the invention, the epitope-presenting carrier used in the methods is an epitope-presenting valency platform molecule, where at least one epitope of the epitope-presenting valency platform molecule specifically binds an anti-dsDNA antibody. Optionally, the epitope-presenting valency platform is a conjugate comprising a non-immunogenic valency platform molecule and two or more double-stranded DNA (dsDNA) epitopes. Exemplary epitope-presenting valency platforms are described below.

In another embodiment, the levels of circulating anti-dsDNA antibodies in a biological fluid of an individual are reduced by contacting the fluid with an epitope (optionally, in the form of an epitope-presenting carrier) ex vivo under conditions that permit the antibodies to bind epitopes on the valency platform. Suitable bodily fluids include those that can be returned to the individual, such as blood, plasma, or lymph. Affinity adsorption apheresis is described generally in Nilsson et al. (1981) Blood 58(1):38-44; Christie et al. (1993) Transfusion 33:234-242; Richter et al. (1997) ASAIO J. 43(1):53-59; Suzuki et al. (1994) Autoimmunity 19: 105-112; U.S. Pat. No. 5,733,254; Richter et al. (1993) Metabol. Clin. Exp. 42:888-894; Richter et al. (1997) ASAIO J. 43(1):53-59; and Wallukat et al. (1996) Int'l J. Card. 54:191-195.

Accordingly, the invention includes methods of reducing levels of SLE-associated antibodies in an individual, comprising treating the individual's blood (including any component thereof which contains antibody) extracoporeally (i.e., outside the body or ex vivo) with an epitope (optionally in the form of an epitope-presenting carrier) under conditions that permit the antibodies to bind the epitope; removing antibody-epitope complexes, if any; and returning the blood to the individual.

In the methods of the invention, the bodily fluid is removed from the individual for extracorporeal binding to an epitope, such as an epitope-presenting valency platform molecule, of this invention. For example, apparatuses and methods for removing blood and separating it into its constituent components are known in the art (see, e.g., U.S. Pat. Nos. 4,086,924; 4,223,672). The blood or portions thereof are then exposed to the valency platform molecule. The valency platform molecule neutralizes (i.e., binds) the unwanted antibody, and the blood components are then returned to the individual.

In a preferred technique, the antibody-valency platform molecule complex (or any complex comprising the antibody) is removed before the fluid is returned to the individual. This may be done, for example, by using a valency platform molecule attached to a solid phase, or by using a soluble valency platform molecule and selectively removing the complex from the treated solution. Valency platform molecules are exemplified; however, as is known in the art, these general principles may be applied to other types of complexes and/or carriers.

To create a solid phase, the valency platform molecule is adapted to render it insoluble. For example, an additional linkage can be added to the valency platform molecule. The linkage is then used to attach the platform to an insoluble structure, such as a polystyrene or polyethylene bead, a polycellulose membrane, or other desirable structure. Commercially available matrices include agarose (a neutral linear polysaccharide generally composed of D-galactose and altered 3,6-anhydrogalactose residues, for example Sepharose™, Pharmacia), activated gels, nitrocellulose, borosilicate, glass fiber filters, silica, polyvinylchloride, polystyrene, and diazotized paper. Methods for preparing peptide-peptide conjugates are described in Hermanson, G. T., “Bioconjugate Techniques”, Academic Press: New York, 1996; and in “Chemistry of Protein Conjugation and Cross-linking” by S. S. Wong, CRC Press, 1993. The biological fluid to be treated is contacted with the solid phase, and antibodies in the fluid complex to the solid phase. The supernatant fluid can then be removed from the solid phase for return to the individual. In some instances, the solid phase can also be cleared of antibody for repeat use by using a suitable wash, providing both the epitope and the valency platform molecule is resistant to the washing solution. Suitable washing solutions may include 0.1 M glycine buffer, pH 2.4, dilute acetic acid, or 1 M KSCN buffered to ˜pH 7.

If the valency platform molecule is not part of a solid phase, then the antibody-carrier complex can be removed from the fluid by any other appropriate method, including but not limited to microfiltration, antibody capture, or precipitation. Solutions suitable to cause precipitation of the complex depend on the solubility of the complex, and may include ammonium sulfate or polyethylene glycol. If the fluid is to be returned to the individual, then the precipitating solution should be chosen so that any that remains in the fluid does not cause an adverse reaction in the individual.

It is understood that the in vivo and ex vivo methods for reducing circulating SLE-associated antibodies described herein may be used in conjunction with each other.

Devices which can be used for reducing the level of antibody in a biological fluid using an epitope (or epitope-presenting carrier) described herein include a flow system, comprising the following elements: a) a port that permits biological fluid to flow into the device; b) a chamber in which the fluid is permitted to contact the epitope-bound valency platform molecule (optionally in a solid phase); c) a port that permits the treated fluid to flow out of the device. Such devices can be designed as continuous flow systems, and as systems that permit the treatment of a single sample from an individual for purposes of analysis or readministration at a subsequent time.

In still another aspect, the invention provides a method of reducing risk of renal flare in an individual with SLE, comprising reducing the levels of circulating anti-dsDNA antibodies in the individual, and maintaining a sustained reduction of the circulating anti-dsDNA antibody of at least about 10% below baseline, wherein the sustained reduction of circulating anti-dsDNA antibodies in the individual results in reduction of incidence of renal flare.

For any of the above methods, the sustained reduction is for at least about one month, at least about two months, at least about three months, at least about four months, at least about five months, at least about six months, at least about one year, or at least about two years or longer. The sustained reduction of circulating anti-dsDNA antibodies of at least about 10% below baseline can be achieved by any methods known in the art and methods described herein.

In another aspect, the invention provides a method of monitoring treatment of renal SLE in an individual, comprising measuring the levels of circulating anti-dsDNA antibodies in the individual, wherein increased levels of circulating anti-dsDNA antibodies are indicative of likelihood of renal flare and sustained decreased levels of circulating anti-dsDNA antibodies of at least about 10% below baseline are indicative of less likelihood of renal flare.

In another aspect, the invention provides methods of monitoring a treatment of renal SLE in an individual, said method comprising measuring level of anti-dsDNA antibody in the individual, wherein a sustained reduction of anti-dsDNA antibody of at least about 10% below baseline indicates effectiveness of the treatment for renal flare.

In another aspect, the invention provides methods of indicating likelihood of success of treatment for renal SLE, said method comprising measuring level of anti-dsDNA antibody in the individual, wherein a sustained reduction of anti-dsDNA antibody of at least about 10% below baseline indicates likelihood of success of the treatment for renal flare.

As sustained reduction of circulating anti-dsDNA antibody in an individual of at least about 10% below baseline correlates with less likelihood of renal flare, monitoring levels of anti-dsDNA antibody may indicate initial responsiveness, efficacy and the appropriate dosage of a treatment. The sustained reduction is generally for at least about one month, at least about two months, at least about three months, at least about four months, at least about five months, at least about six months, at least about one year, at least about two years or longer. It is understood that monitoring treatment means that biological sample(s) are obtained at different times, for example, before application of treatment, during application of treatment, and the level of circulating anti-dsDNA antibodies are measured and compared, either with each other, a control, and/or a desired value. The biological sample(s) can be obtained once a week, once every two weeks, once a month, once every two months, and/or once every three months. The level of circulating anti-dsDNA antibody can be measured by methods known in the art, such as Farr assay. See, for example, ter Borg et E J, et al. (1990) Arthritis Rheum. 33:634-43; Alarcon-Segovia D, et al. (2003) Arthritis Rheum. 48:442-454.

With the above methods, as this disclosure makes clear, the percentage of sustained reduction may be for increasing percentage (which may be adjusted over time) and/or varying lengths of time.

In some embodiments, the methods of the invention include any one or more of the following steps: (a) comparison of anti-dsDNA antibody level measurement to another anti-dsDNA antibody level measurement; (b) comparison of anti-dsDNA antibody level measurement to baseline; (c) determination or assessment of whether a comparison between anti-dsDNA antibody level measurement to baseline is at least about 10% reduction (in some embodiments, an increase); (d) determination of percentage reduction (or increase) of anti-dsDNA antibody level as compared to another anti-dsDNA antibody level (such as baseline); (e) adjusting dosage based on the level of anti-dsDNA antibody to achieve at least about 10% reduction. Any of these steps are generally performed with respect the a given individual.

IV. Selection of Individuals for Treatment

Individuals for treatment are identified or indicated by any of a number of criteria. One criterion is diagnosis of SLE, or if SLE is suspected. Individuals especially suitable for treatment are human. Individuals suitable for treatment have, have had, and/or are at risk of renal SLE disease and/or Major SLE flare. In some embodiments, individuals suitable for treatment have antibodies with high affinity to a dsDNA epitope (for example, LJP 394), and/or have significant renal impairment.

The instant method involves treating and/or selecting an individual who has, or is suspected of having, systemic lupus erythematosus (SLE) who also has or is at risk of renal disease and/or Major SLE flare. In some embodiments, the individual has impaired renal function. The symptoms of SLE are well known in the art, and it is well within the knowledge of those of ordinary skill in the art to identify individuals having, or who are suspected of having, SLE. Within the group of individuals having, or being suspected of having, SLE, selecting those having renal disease (including significantly impaired renal function) may be on the basis of any clinical indication of renal impairment known in the art, including, but not limited to, anuria, oliguria, elevated serum creatinine levels, elevated BUN, proteinuria, hematuria (occult or gross), reduced creatinine clearance, impaired glomeral filtration, renal pathology following biopsy and the like. As will be apparent to one of skill in the art, a diagnosis of renal dysfunction, such as a diagnosis of subacute glomerulonephritis, nephrotic syndrome, or mild to severe nephritis, will also identify a patient with renal disease and thus serve as a basis for treating that individual and/or selection of the individual for treatment in accordance with the instant methods.

As will be apparent, the quantitative level of a particular clinical parameter that indicates renal disease (including a significant impairment of renal function) will depend on the particular clinical parameter. Proteinuria is easily detected at a ‘screening’ level using colorimetric “dipstick” testing of urine, and can be followed up by more sensitive and accurate laboratory testing. Preferably, when the presence of a renal disease is identified by proteinuria, an individual is considered to have renal disease when at least about 500 mg of protein is excreted in the urine per day, more preferably at least about (i.e., greater than or equal to about) 1.5, 2, 2.5, 3, 3.5, 5.0, 6.0, 7.0, 8.0, 9.0, or 10 grams of protein per day. When serum creatinine is used as the indicator of renal disease, an individual will be considered to have renal disease when serum creatinine levels are at least about (i.e., greater than or equal to about) 1.5, 2, 2.5 3.0, 3.5, 4.0, 5.0, 6.0, 7.0, 8.0, 9.0, or 10 milligrams per deciliter (mg/dL). In some embodiments, the serum creatinine levels are at least about 1.5 mg/dL.

In some embodiments, anti-dsDNA antibodies from individual bind with high affinity of the treatment modality (such as dsDNA epitope) and affinity is used as a basis for selecting the individual for treatment (receive and/or continue to receive treatment).

As will be understood by one of skill in the art, administration of an effective amount of a dsDNA epitope, preferably in the form of an epitope-presenting carrier, and preferably wherein at least one of the epitopes is bound at a high initial affinity by antibodies from the patient entails assessing antibody affinity from an individual in those embodiments in which selection is based on antibody affinity, wherein said individual has, or is suspected of having, SLE. For purposes of this invention; (a) the affinity in question is with respect to an individual's antibodies, that is, antibodies obtained from that individual; (b) the antibody for which affinity is measured is an antibody associated with, and/or implicated in SLE; and (c) the binding of interest is binding of antibody to an epitope which binds to the antibody(ies), generally the epitope to be used in the proposed treatment, as described herein (i.e., a dsDNA epitope), or binding which correlates with binding of the epitope(s) to be used in the proposed treatment.

For all embodiments of the invention which use or are directed to K_{D}′, whether screening, treatment, monitoring, or any other methods directed to assessing affinity, it is understood that other, equivalent values can be measured and used, and are encompassed by this invention. For example, as discussed below, there are a number of methods known in the art which can measure (and express) affinity of antibodies from an individual for an epitope to be used for treatment (in the context of this invention, a double stranded DNA epitope). As is understood and conveyed by this disclosure, affinity may be measured using any epitope whose binding to the dsDNA antibody correlates with binding of the epitope(s) to be used in the proposed treatment (for example, a single-stranded counterpart of a double-stranded polynucleotide). K_{D}′ is one of these parameters, and equivalent parameters can be measured and used in this invention. Further, with respect to K_{D}′ cut-off values reported herein, the basis of this finding was administering about 100 mg of LJP 394 conjugate about once a week.

Measurement of affinity, either represented by measuring K_{D}′ or by some other method, either before or during treatment is strong, if not conclusive, indication that this parameter was a basis for selecting the individual to receive (and/or continue to receive) treatment. Accordingly, with respect to all treatment methods described herein, and as the definition for “is used as a basis” states, other embodiments include (1) assessing, or measuring, the affinity as described herein (and preferably selecting an individual suitable for receiving (including continuing to receive) treatment); and (2) administering the treatment(s) as described herein. As described herein, in some embodiments, more than one measurement is made, when change (if any) in affinity is assessed.

Antibody affinity may be measured using methods known in the art which assess degree of binding of a DNA epitope to an antibody. Generally, these methods comprise competition assays and non-competition assays. With respect to polynucleotide epitopes (which may be used in an epitope-presenting carrier), affinity may be measured using polynucleotide alone or polynucleotide-containing epitope-presenting carriers (as long as the polynucleotide and epitope-presenting carrier give equivalent, or at least convertible, values). Affinity may be measured using the epitope (or a molecule or moiety comprising the epitope) used in the epitope-presenting carrier; alternatively, a similar, non-identical epitope may be used, as long as its affinity may be at least correlated to the affinity of the epitope used in the conjugate, so that a meaningful measurement of affinity may be obtained.

In a competition assay, varying concentrations of antibody or epitope are reacted with epitope or antibody, and results may be expressed in terms of amount of antibody (generally in terms of concentration) required to reach half-maximal binding, generally designated as IC₅₀.

Another convenient way to express affinity is apparent equilibrium dissociation constant, or K_{D}′, which reflects the titer-weighted average affinity of the antibody for the antibody-binding epitope or epitope-presenting carrier. Antibody is generally obtained from whole blood and measured, by plasma, serum, or as an IgG fraction, and the affinity of this fraction for the epitope or epitope-presenting carrier is measured. Methods of obtaining IgG fractions are known in the art and are described herein. One preferred way to measure affinity is to measure K_{D}′ based on a surface plasmon resonance assay.

Another way to measure affinity is by kinetic (i.e., non-equilibrium) analysis, methods of which are known in the art. Preferably, rate of dissociation (i.e., off rate) of antibody from epitope is measured.

In preferred embodiments, the affinity of the individual's antibodies for the dsDNA epitope(s) (whether measured directly using the epitope itself or using a moiety/epitope the affinity of which may be correlated to the affinity of the epitope used in the carrier) is measured as the apparent equilibrium dissociation constant (K_{D}′) for the dsDNA epitope(s) in the carrier before or upon initiation of treatment is less than about (in some embodiments, less than or equal to about) 1.0 mg IgG per mL. In other embodiments, the K_{D}′ is less than about (in some embodiments, less than or equal to about) any of the following: 0.8; 0.7; 0.6; 0.5; 0.4; 0.3; 0.2; 0.1; 0.09; 0.08; 0.07; 0.06; 0.05; 0.025. In some embodiments, K_{D}′ is less than about (in some embodiments, less than or equal to about) 0.8 mg IgG per mL. In some embodiments, K_{D}′ is less than or equal to about (in some embodiments, less than or equal to about) 0.5 mg IgG per mL. In some embodiments, K_{D}′ is less than about (in some embodiments, less than or equal to about) 0.1 mg IgG per mL. In some embodiments, the dsDNA epitope used comprises, consists essentially of, or consists of the double-stranded polynucleotide 5′-GTGTGTGTGTGTGTGTGTGT-3′ (SEQ ID NO:1) in combination with its complementary strand, particularly the sequence 3′-CACACACACACACACACACA-5′ (SEQ ID NO:2), or one of the single-stranded polynucleotides 5′-GTGTGTGTGTGTGTGTGTGT-3′ (SEQ ID NO:1) or 3′-CACACACACACACACACACA-5′ (SEQ ID NO:2), and the initial K_{D}′ is less than about 0.8 mg IgG per ml (in some embodiments, less than or equal to 0.8 mg IgG per ml). In some embodiments, the therapeutic moiety is LJP 394.

In some embodiments, an individual is considered to have high affinity for a dsDNA epitope if the antibody affinity of the individual is in a relatively high percentile ranking of affinity compared to a population. For example, there is a range of antibody affinities over a given patient population, and individuals considered to have high affinity for a dsDNA epitope can be identified based on a percentile ranking of antibody affinity with respect to this population. Accordingly, in some embodiments, an individual is considered to have high affinity antibodies if the antibody affinity relative to the dsDNA epitope(s) for that individual is greater than about the 20th percentile (i.e., in about the top 80% of affinities for that population), and considered to not have high affinity antibodies (i.e., is not selected for treatment in accordance with the invention) if the individual's antibody affinity is in or below the 20th percentile. In other embodiments, an individual is included in treatment, or identified as suitable to receive treatment, if the antibody for that individual is greater than about the 50th percentile for that population. In some embodiments, the individual is considered to have high affinity antibody if the affinity is greater than the 70th, 75th, 80th, 85th, 90th, or 95th percentile. A population may be about, or alternatively at least about any of the following, in terms of number of individuals measured: 10, 15, 20, 25, 30, 50, 60, 75, 100, 125, 150, 175, 200, 225, 250, 300, 400, 500. Preferably, a sufficient number of individuals are measured to provide a statistically significant population, which can be determined by methods known in the art. An upper limit of a population may be any number, including those listed.

Affinity may or may not change over the course of treatment. In some embodiments which include a step wherein the individual's antibody affinity for the dsDNA epitope(s) is remeasured after initiation of the treatment, the treatment may be continued if the average affinity of the individual's antibodies for the dsDNA epitope(s) is decreased by at least about 15%, preferably at least about 20%, more preferably at least about 25%, more preferably at least about 40%, more preferably at least about 50%, compared to the affinity measured before or at initiation of treatment, or may be discontinued if the antibody affinity has not decreased by at least about 15% (preferably at least about 20%, more preferably at least about 25%, more preferably at least about 40%, more preferably at least about 50%). For these embodiments, antibody affinity is measured after initiation of treatment (for comparison to antibody affinity before or upon initiation of treatment) at least about 4 weeks, preferably at least about 6 weeks, more preferably at least about 10 weeks, more preferably at least about 12 weeks, after initiation of treatment. In other embodiments, treatment may be continued if antibody affinity is decreased at least about any of the following (as compared to antibody affinity before or upon initiation of treatment): 40%, 50%, 75%, 100%, 200%, 500%. Preferably, antibody affinity is measured as the K_{D}′. As is understood by those of skill in the art, K_{D}′ values are inversely proportional to the affinity of the antibodies measured. Accordingly, in some embodiments, when K_{D}′ values are used to measure antibody affinity, treatment may be continued if the K_{D}′ increases by at least about 15%, and may be continued if K_{D}′ is increased at least about any of the following (as compared to antibody affinity before or upon initiation of treatment): 40%, 50%, 75%, 100%, 200%, 500%.

When antibody affinity is assayed using surface plasmon resonance, a reduction in affinity of at least about 15%, preferably at least about 20%, more preferably at least about 25%, more preferably at least about 40%, more preferably at least about 50% indicates responsiveness and that continuation of the treatment is indicated. For a competitive Farr assay, the same reductions in affinity generally apply. For other assays, the change can be at least about any of the above percentages, and further can be at least about any of the following percentages: 75%, 100%, 150%, 200%, 250%, 300%, 350%, 400%, 450%, 500%.

These methods may be practiced independently of the treatment methods, and may be practiced by a skilled technician other than a medical doctor, using equipment and/or techniques of the art.

V. Administration of Epitopes, Including Epitope-Presenting Carriers

Various formulations of ds-DNA epitopes or epitope-presenting carriers comprising these epitopes, such as epitope-presenting valency platform molecules, may be used for administration, and, as such, the methods of this invention include administering a composition comprising any ds-DNA epitope, epitope-presenting carrier, or epitope-presenting valency platform molecule(s) described herein. In some embodiments, the compositions may be administered “neat” (e.g., dissolved in pure water, such as USP water for injection). In some embodiments, the compositions comprise a dsDNA epitope or a dsDNA epitope-presenting carrier (s) and a pharmaceutically acceptable excipient, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington's Pharmaceutical Sciences 19th Ed. Mack Publishing (1995) and Remington: The Science and Practice of Pharmacy, 20th Ed., Lippincott, Williams & Wilkins (2000).

Generally, these compositions are formulated for administration by injection (e.g., intraperitoneally, intravenously, subcutaneously, intramuscularly, etc.). Accordingly, these compositions are preferably combined with pharmaceutically acceptable vehicles such as saline, Ringer's solution, dextrose solution, and the like, and, as is understood in the art, are usually sterile to be suitable for injection, especially in humans. Generally, the epitope or epitope-presenting carrier will normally constitute about 0.01% to 10% by weight of the formulation due to practical, empirical considerations such as solubility and osmolarity. The particular dosage regimen, i.e., dose, timing and repetition, will depend on the particular individual and that individual's medical history. Generally, a dose of about 1 μg to about 100 mg conjugate/kg body weight, preferably about 100 μg to about 10 mg/kg body weight, preferably about 150 μg to about 5 mg/kg body weight, preferably about 250 μg to about 1 mg conjugate/kg body weight. Empirical considerations, such as the half life, generally will contribute to determination of the dosage. Other dosages, such as about 50 to 100 mg per week, 50 to 250 mg per week, and 50 to 500 mg per week (with any value inbetween the lower and upper limit of these ranges) are also contemplated. Examples 2 and 3 provide examples of two dosing regimens. If used as a toleragen, conjugate may be administered daily, for example, in order to effect antibody clearance (pheresis), followed by less frequent administrations, such as two times per week, once a week, or even less frequently. Frequency of administration may be determined and adjusted over the course of therapy, and is based on maintaining tolerance (i.e., reduced or lack of immune response to dsDNA). Other appropriate dosing schedules may be as frequent as continuous infusion to daily or 3 doses per week, or one dose per week, or one dose every two to four weeks, or one dose on a monthly or less frequent schedule depending on the individual or the disease state. Repetitive administrations, normally timed according to B cell turnover rates, may be required to achieve and/or maintain a state of humoral anergy. Such repetitive administrations generally involve treatments of about 1 μg to about 10 mg/kg body weight or higher every 30 to 60 days, or sooner, if an increase in anti-dsDNA antibody level is detected. Alternatively, sustained continuous release formulations of the compositions may be appropriate. Various formulations and devices for achieving sustained release are known in the art.

In some embodiments, LJP 394, a dsDNA epitope presenting valency platform molecule described below, is formulated as a sterile, colorless liquid in an isotonic phosphate-buffered saline solution for intravenous (IV) administration. Each 1 mL of solution contains 50 mg of LJP 394, 1.9 mg Na2HPO4*7H20, 0.30 mg NH₂PO₄*H₂0, and 5.8 mg NaCl in water for Injection, USP (pH 6.8 -8.0). The formulation contains no preservatives. Other formulations are designed to be 20 mg/mL, 10 mg/mL, and 1 mg/mL of LJP 394. The formulations are preferably stored at cooler temperatures, such as 2 to 8° C. In other embodiments, each 1 mL of solution contains 50 mg of LP 394, 1.9 mg Na₂HPO₄ *7H₂0, 0.30 mg NH₂PO₄*H₂0, and 8.0 mg NaCl in water for Injection, USP (pH 6.8 -8.0). LJP 394 is also optionally administered as 100 mg in 2 ml. As described herein, LJP 394 may be administered 100 mg weekly.

Due to the chronic nature of systemic lupus erythematosus, the epitopes, epitope-presenting carriers, and epitope-presenting valency platform molecules, including conjugates, of the present invention will in some embodiments be administered to patients for extended periods of time. For instance, in one embodiment, the conjugate comprising a non-immunogenic valency platform molecule and two or more double-stranded DNA epitopes is administered to an individual for treatment of renal SLE.

Other formulations include those suitable for oral administration, which may be suitable if the conjugate is able to cross the mucosa. Similarly, an aerosol formulation may be suitable.

Other formulations include suitable delivery forms known in the art including, but not limited to, carriers such as liposomes. Mahato et al. (1997) Pharm. Res. 14:853-859. Liposomal preparations include, but are not limited to, cytofectins, multilamellar vesicles and unilamellar vesicles.

In some embodiments, more than one epitope or epitope-presenting carrier may be present in a composition. Such compositions may contain at least one, at least two, at least three, at least four, at least five different epitopes or epitope-presenting carriers. Such “cocktails”, as they are often denoted in the art, may be particularly useful in treating a broader range of population of individuals. They may also be useful in being more effective than using only one (or fewer than are contained in the cocktail) dsDNA epitope or dsDNA epitope-presenting carrier.(s).

The compositions may be administered alone or in conjunction with other forms of agents that serve to enhance and/or complement the effectiveness of a epitope or epitope-presenting carrier of the invention, including, but not limited to, anti-T cell treatments. Such treatments usually employ agents that suppress T cells such as steroids or cyclosporin. Other agents are corticosteroid and/or cyclophosphamide immunosuppressive therapy. Other possible agents which may be administered in combination with the epitopes, epitope-presenting carriers, or epitope-presenting valency platform molecules are psychiatric medications, such as antidepressants.

VI. Treatment Modalities

Any agent which can effect sustained reduction of anti-dsDNA antibodies is suitable for this invention. More desirably, an agent which selectively reduces anti-dsDNA antibodies is used. In some embodiments, the agent is an immunosuppressive drug, such as high dose corticosteroids (HDCC) and/or cyclophosphamide. In some embodiments, the agent is an anti-idiotype antibody to anti-dsDNA antibodies.

A. Epitopes

Epitopes used in the methods of the present invention comprise dsDNA epitopes.

Double-stranded DNA (dsDNA) epitopes for use in the methods of the present invention may be any chemical moiety which specifically binds to a dsDNA antibody. In particular, epitopes of interest include those that bind the anti-polynucleotide (particularly anti-DNA, including anti-double stranded DNA) antibodies that occur in systemic lupus erythematosus. Generally, but not necessarily, the dsDNA epitopes used are polynucleotides, optionally DNA (including DNA analogs), and optionally double-stranded DNA or optionally single-stranded DNA. Polynucleotide epitopes (including polynucleotide comprising such epitopes) may be single-stranded, double-stranded, or may be partially single or double stranded. Epitopes also include mimetics and analogs of single-stranded, double-stranded, or may be partially single or double stranded polynucleotide. In some embodiments, the epitope is a polypeptide. In some embodiments, the epitope is a peptide.

Examples of suitable epitopes are described, for instance, in U.S. Pat. Nos. 5,162,515; 5,391,785; 5,276,013; 5,786,512; 5,726,329; 5,552,391; 5,268,454; 5,633,395; 5,606,047).

For instance, optionally, the polynucleotide is double-stranded DNA. In some embodiments, the polynucleotide comprises, consists essentially of, or consists of the double-stranded sequence 5′-GTGTGTGTGTGTGTGTGTGT-3′ (SEQ ID NO:1) in combination with the complementary polynucleotide sequence, particularly the sequence 3′-CACACACACACACACACACA-5′ (SEQ ID NO:2).

In some embodiments, the polynucleotide is single-stranded DNA comprising, consisting essentially of, or consisting of the sequence 5′-GTGTGTGTGTGTGTGTGTGT-3′ (SEQ ID NO:1). In some alternative embodiments, the polynucleotide is single-stranded DNA comprising, consisting essentially of, or consisting of the sequence 3′-CACACACACACACACACACA-5′ (SEQ ID NO:2). In another embodiment, the dsDNA epitope comprises, consists essentially of, or consists of the pentapeptide sequence Asp/Glu-Trp-Asp/Glu-Tyr-Ser/Gly.

The suitability of particular epitopes for binding antibodies according to this invention can be identified and/or confirmed using techniques known in the art and described herein. For example, to select the optimum epitope from a library of small drug molecules believed to mimic the dsDNA epitope for SLE, a family of platforms can be constructed in which each of the candidates is alternatively displayed on a similar platform molecule. The composition is then tested for efficacy. For example, for in vivo use, an animal model is used in which there are circulating anti-DNA antibodies, such as, for example, the BXSB mouse model system. The animals can be immunized with an appropriate epitope to initiate the antibody response, if necessary. Test candidates assembled onto a platform are then used to treat separate animals, either by administration, or by ex vivo use, according to the intended purpose. The animals are bled before and after treatment, and the antibody levels in plasma are determined by standard immunoassay as appropriate for the specific antibody. Efficacy of the candidates is then assessed according to antibody affinity assays designed to indicate antibodies specific for the epitope being tested. Appropriate affinity assays are described herein.

Polynucleotides (or other dsDNA epitope candidates such as polypeptides) may be screened for binding activity with antisera containing the antibodies of interest, for example, SLE antisera, by the assays known in the art. Examples of such assays include competitive affinity assays, for example, a competitive Farr assay and/or a competitive ELISA assay, and/or non-competitive, equilibrium affinity assay, such as the surface plasmon resonance (for example, using BIACORE®) based assay as known in the art and as described herein and in WO 01/41813.

Antibody affinity may be measured using methods known in the art which assess degree of binding of DNA epitope to antibody. Generally, these methods comprise competition assays and non-competition assays. With respect to polynucleotide epitopes (which will be used in a carrier to be administered), affinity may be measured using polynucleotide alone or polynucleotide-presenting carriers (as long as the polynucleotide and conjugate or carrier give equivalent, or at least convertible, values).

A competitive Farr assay is an exemplary assay. In a competition assay, varying concentrations of antibody or epitope are reacted with epitope or antibody, and results may be expressed in terms of amount of antibody (generally in terms of concentration) required to reach half-maximal binding, generally designated as IC₅₀- Polynucleotide duplexes having an IC₅₀ of less than about 500 nM, preferably less than 50 nM, are deemed to have significant binding activity and are, therefore, useful for making the epitope-presenting carriers of this invention.

Another convenient way to express affinity is apparent equilibrium dissociation constant, or K_{D}′, which reflects the titer-weighted average affinity of the antibody for the antibody-binding epitope on the carrier. Antibody is generally obtained from whole blood and measured, by plasma, serum, or as an IgG fraction, and the affinity of this fraction for the epitope-presenting carrier is measured. Methods of obtaining IgG fractions are known in the art and are described herein. One preferred way to measure affinity is to measure K_{D}′ based on a surface plasmon resonance assay.

Another way to measure affinity is by kinetic (i.e., non-equilibrium) analysis, methods of which are known in the art. Preferably, rate of dissociation (i.e., off rate) of antibody from epitope is measured.

In one embodiment the apparent equilibrium dissociation constant (K_{D}′) for each of the double-stranded DNA epitopes with respect to the antibody to which it specifically binds is less than about 1.0 mg IgG per ml. In some other embodiments of the invention the apparent equilibrium dissociation constant (K_{D}′) for each of the double-stranded DNA epitopes with respect to the antibody to which it specifically binds is less than about 0.8 mg IgG per ml, less than about 0.5 mg IgG per ml, or less than about 0.2 mg IgG per ml. In other embodiments, the K_{D}′ is less than or equal to about any of these values.

It is understood that, for purposes of this invention, more than one type of dsDNA epitope(s) may be used in preparing an epitope-presenting carrier. Alternatively, one type (i.e., one chemical species) of a dsDNA epitope may be used. If a polynucleotide (such as dsDNA) is used, generally the length is greater than about 10 base pairs (bp), more preferably greater than about 15 bp, more preferably greater than or equal to about 20 bp. Generally, but not necessarily, the length is less than about 1 kb, preferably less than about 500 bp, preferably less than about 100 bp. It is understood that these values also pertain to single-stranded forms or partially double-stranded forms.

B. Epitope-Presenting Carriers

In some embodiments, the epitope-presenting carrier comprises at least one attached or bound epitope. Embodiment comprises (some embodiments, consist of) the epitope. In one embodiment, the dsDNA epitope administered to an individual with SLE in any of the methods described herein is administered in the form of an epitope-presenting carrier.

Any of a variety of carriers may be used, as long as the carrier does not elicit an undesirable or unacceptable immune response. The carrier may be any chemical moiety, and have any chemical structure, including, but not limited to, organic and inorganic molecules, polypeptides (i.e., polymers of amino acids), nucleic acids, carbohydrates, other polymers, artificial structures, and lipid structures (such as liposomes or micelles) made by standard techniques, or polymerized as described in U.S. Pat. No. 5,512,294.

In one embodiment, the epitope-presenting carrier comprises more than one attached or bound epitopes. Optionally, the epitope-presenting carrier is an epitope-presenting valency platform molecule. Exemplary epitope-presenting valency platform molecules are described below.

Epitope-presenting carrier may also be a monoclonal antibody (such as a humanized antibody) to a complement receptor on red blood cells (e.g., CR-1) cross-linked to polynucleotide epitopes including double stranded DNA, single stranded DNA. These conjugates can be used to reduce autoantibodies to dsDNA by carrying the autoantibodies to liver for destruction. See, U.S. Pat. No. 5,879,679; U.S. Pub. No. 20020103343; WO 95/22977; Lindorfer et al., J. Immunol. Methods. 248:125-38 (2001); Picus et al., Clinical Immunol. 105:141-154 (2002); Taylor et al. J. of Hematogherapy 4:357-362 (1995).

Other carriers are described in U.S. Pat. Nos. 6,022,544; 6,340,460; 6,375,951; U.S. Pub. No. 2002/0187156.

C. Epitope-Presenting Valency Platform Molecules

In one embodiment, epitope-presenting valency platform molecule are used in the methods of the invention. In one embodiment, the epitope-presenting valency platform molecule is a conjugate comprising a non-immunogenic valency platform molecule and at least two (i.e., two or more) dsDNA epitopes, optionally polynucleotides which bind to anti-dsDNA antibody from the individual.

Any of a variety of non-immunogenic valency platform molecules (also called “platforms”) may be used in the conjugates of the invention. Many have been described in the art, such as polymers, and need not be described herein. Any non-immunogenic, acceptably low to non-toxic molecule which provides requisite attachment sites such that the conjugate may act to bind circulating anti-ds DNA antibody and/or induce B cell anergy and/or apoptosis in cells producing these antibodies may be used. Preferably, the conjugates comprise a chemically defined valency platform molecule in which a precise valency (as opposed to an average) is provided. Accordingly, a defined valency platform is a platform with defined structure, thus a defined number of attachment points and a defined valency. Certain classes of chemically defined valency platforms, methods for their preparation, conjugates comprising them and methods for the preparation of such conjugates suitable for use within the present invention include, but are not limited to, those described in the U.S. Patents Nos. 5,162,515; 5,391,785; 5,276,013; 5,786,512; 5,726,329; 5,268,454; 5,552,391; 5,606,047; 5,663,395; and 6,060,056; and in commonly-owned U.S. Ser. Nos. 60/111,641 (U.S. Ser. No. 09/457,607, U.S. Pat. No. 6,458,953, and PCT App. No. PCT/US99/29339); 60/138,260 (U.S. Ser. No. 09/590,592 and PCT App. No. PCT/US00/15968, WO 00/75105), U.S. 09/457,913 (U.S. Pat. No. 6,399,578, PCT App. No. PCT/US99/29338, WO 00/34231), U.S. 09/457,607 (U.S. Pat. No. 6,458,953, PCT/US99/29339) and U.S. 09/877,387 (PCT/US01/18446, WO 01/93914), all of which are hereby incorporated by reference.

A platform may be proteinaceous or non-proteinaceous (i.e., organic). Examples of proteinaceous platforms include, but are not limited to, albumin, gammaglobulin, immunoglobulin (IgG) and ovalbumin. Borel et al. (1990) Immunol. Methods 126:159-168; Dumas et al. (1995) Arch. Dematol. Res. 287:123-128; Borel et al. (1995) Int. Arch. Allergy Immunol. 107:264-267; Borel et al. (1996) Ann. N.Y. Acad. Sci. 778:80-87.

The valency of a chemically-defined valency platform molecule within the present invention can be predetermined by the number of branching groups added to the platform molecule. Suitable branching groups are typically derived from diamino acids, triamines, and amino diacids.

Preferred valency platform molecules are biologically stabilized, i.e., they exhibit an in vivo excretion half-life often of hours to days to months to confer therapeutic efficacy, and are preferably composed of a synthetic single chain of defined composition. They generally have a molecular weight in the range of about 200 to about 200,000, preferably about 200 to about 50,000 (or less, such as 30,000). Examples of valency platform molecules within the present invention are polymers (or are comprised of polymers) such as polyethylene glycol (PEG), poly-D-lysine, polyvinyl alcohol, polyvinylpyrrollidone, D-glutamic acid and D-lysine (in a ratio of 3:2). Preferred polymers are based on polyethylene glycols (PEGs) having a molecular weight of about 200 to about 8,000, or, in some embodiments, about 200 to about 10,000. In other embodiments, the molecular weight can range between about 40,000 to about 100,000; with a range of about 10,000 to about 20,000 as preferable. Other suitable platform molecules for use in the conjugates of the invention are albumin and IgG. Valency platform molecules should be of a size such that a conjugate made with the valency platform does not become a T cell independent immunogen.

Valency platform molecules suitable for use within the present invention are the chemically-defined valency platform molecules disclosed, for example, in co-owned U.S. Pat. No. 5,552,391, hereby incorporated by reference. These platforms generally have low polydispersity. Homogeneous chemically-defined valency platform molecules suitable for use within the present invention are derivatized 2,2′-ethylenedioxydiethylamine (EDDA) and triethylene glycol (TEG). The AHAB-TEG platform used for LJP 394 (a monodisperse platform) is described below.

In some embodiments, the valency platform molecules have the advantage of having a substantially homogeneous (i.e., uniform) molecular weight (as opposed to polydisperse molecular weight). Accordingly, a population of these molecules (or conjugates thereof) are substantially monodisperse, i.e., have a narrow molecular weight distribution. A measure of the breadth of distribution of molecular weight of a sample of a platform molecule (such as a composition and/or population of platform molecules) is the polydispersity of the sample. Polydispersity is used as a measure of the molecular weight homogeneity or nonhomogeneity of a polymer sample. Polydispersity is calculated by dividing the weight average molecular weight (Mw) by the number average molecular weight (Mn). The value of Mw/Mn is unity for a perfectly monodisperse polymer. Polydispersity (Mw/Mn) is measured by methods available in the art, such as gel permeation chromatography. The polydispersity (Mw/Mn) of a sample of valency molecules is preferably less than about 2, more preferably, less than about 1.5, or less than about 1.2, less than about 1.1, less than about 1.07, less than about 1.02, or, e.g., about 1.05 to 1.5 or about 1.05 to 1.2. Typical polymers generally have a polydispersity of about 2-5, or in some cases, 20 or more. Advantages of the low polydispersity property of these valency platform molecules include improved biocompatibility and bioavailability since the molecules are substantially homogeneous in size, and variations in biological activity due to wide variations in molecular weight are minimized. The low polydispersity molecules thus are pharmaceutically optimally formulated and easy to analyze. Accordingly, in some embodiments, the valency platform molecules have very low polydispersity, and, in some embodiments are monodisperse.

Other suitable platforms for dsDNA epitopes are tetrabromoacetyl compounds, and other tetravalent and octavalent valency platform molecules, such as those described in Jones et al. (1995) J. Med Chem. 38:2138-2144; and U.S. Patent references provided above.

Additional suitable valency platform molecules include, but are not limited to, tetraaminobenzene, heptaaminobetacyclodextrin, tetraaminopentaerythritol, 1,4,8,11-tetraazacyclotetradecane (Cyclam) and 1,4,7,10-tetraazacyclododecane (Cyclen).

In some embodiments, a platform having a defined number of attachment sites also comprises a (one or more) polyethylene oxide group, as described, for example, in U.S. patents and patent applications described above as well as U.S. Ser. No. 09/877,387, filed Jun. 7, 2001 (PCT/US01/18446). The molecular weight of PEG can be any molecular weight, including, but not limited to, greater than about 200, 500, 1000, 2000, 5000, 10,000, 15,000, 18,000, 22,000, 40,000, 50,000, 80,000, 100,000 Daltons. In one embodiment, in the valency platform molecule, the high molecular weight polyethylene oxide group has the formula:
—(CH2CH2O)n-

wherein n is greater than 500; n is greater than 400; n is greater than 500; n is greater than 600; n is greater than 700; or n is greater than 800. In another embodiment, the valency platform molecule comprises a core group and at least three arms wherein each arm comprises a terminus. The core group and/or the arms may comprise a high molecular weight polyethylene oxide group. The high molecular weight polyethylene oxide group also may be attached to the core or arm. In some embodiments, a composition comprising the valency platform molecules is provided, wherein the molecules have a polydispersity less than 1.2. In another embodiment, the valency platform molecule may comprise at least three reactive conjugating groups such as hydroxyl, thiol, isocyanate, isothiocyanate, amine, alkyl halide, alkylmercurial halide, aldehyde, ketone, carboxylic acid halide, α-halocarbonyl, α,β-unsaturated carbonyl, haloformate ester, carboxylic acid, carboxylic ester, carboxylic anhydride, O-acyl isourea, hydrazide, maleimide, imidate ester, sulfonate ester, sulfonyl halide, α,β-unsaturated sulfone, aminooxy, semicarbazide, or β-aminothiol. In another embodiment, the valency platform molecule comprises at least 3 aminooxy groups and/or at least 3 carbamate groups.

In general, these platforms are made by standard chemical synthesis techniques. PEG must be derivatized and made multivalent, which is accomplished using standard techniques. Some substances suitable for conjugate synthesis, such as PEG, albumin, and IgG are available commercially.

For purposes of this invention, the valency platform molecules have a minimum valency of at least two, preferably at least four, preferably at least six, more preferably at least eight, preferably at least 10, preferably at least 12. As an upper limit, valency is generally less than 128, preferably less than 64, preferably less than 35, preferably less than 30, preferably less than 25, preferably less than 24, preferably less than 20, although the upper limit may exceed 128. Conjugates may also have valency of ranges of any of the lower limits of 2, 4, 6, 8, 10, 12, 16, with any of the upper limits of 128, 64, 35, 30, 25, 24, 20.

In some embodiments, the valency platform molecule comprises a carbamate linkage, i.e., —O—C(═O)—N<). Such platforms are described in a co-owned patent application entitled “Valency Platform Molecules Comprising Carbamate Linkages” U.S. Ser. No. 60/111,641 (U.S. Ser. No. 09/457,607, U.S. Pat. No. 6,458,953, and PCT App. No. PCT/US99/29339), hereby incorporated by reference. Other valency platform molecules are described in the co-owned patent application entitled “Multivalent Platform Molecules Comprising High Molecular Wight Polyethylene Oxide,” U.S. Ser. No. 09/877,387 (U.S. Publication No. 2002/0110535).

In other embodiments, valency platforms may be used which, when conjugated, provide an average valency (i.e., these platforms are not precisely chemically defined in terms of their valency). Examples of such platforms are polymers such as linear PEG; branched PEG; star PEG; polyamino acids; polylysine; proteins; amino-functionalized soluble polymers.

In some embodiments, the conjugates include branched, linear, block, and star polymers and copolymers, for example those comprising polyoxyalkylene moieties, such as polyoxyethylene molecules, and in particular polyethylene glycols. The polyethylene glycols preferably have a molecular weight less than about 10,000 daltons. In one embodiment, polymers with low polydispersity may be used. For example, polyoxypropylene and polyoxyethylene polymers and copolymers, including polyethylene glycols may be modified to include aminooxy groups, wherein the polymers have a low polydispersity, for example, less than 1.5, or less than 1.2 or optionally less than 1.1 or 1.07. Preferably, the polymers comprise at least 3 aminooxy groups, or at least 4, 5, 6, 7, 8, or more.

D. Conjugation of Epitope(s) with Carriers

Conjugation of a biological or synthetic molecule to a carrier, such as a valency platform molecule, may be effected in any number of ways including covalent and non-covalent, typically involving one or more crosslinking agents and functional groups on the biological or synthetic molecule and valency platform molecule. Examples of standard chemistry which may be used for conjugation include, but are not limited to: 1) thiol substitution; 2) thiol Michael addition; 3) amino alkylation (reductive alkylation of amino groups); 4) disulfide bond formation; 5) acylation of amines. Linkage can be direct or indirect.

In some embodiments and as an example, the synthetic polynucleotide duplexes that are coupled to a carrier, such as a valency platform molecule, are composed of at least about 20 bp and preferably 20-50 bp. Polynucleotides described herein are deoxyribonucleotides unless otherwise indicated and are set forth in 5′ to 3′ orientation. Preferably the duplexes are substantially homogeneous in length; that is, the variation in length in the population will not normally exceed about ±20%, preferably ±10%, of the average duplex length in base pairs. They are also preferably substantially homogeneous in nucleotide composition; that is, their base composition and sequence will not vary from duplex to duplex more than about 10%. Most preferably they are entirely homogeneous in nucleotide composition from duplex to duplex.

Based on circular dichroic (CD) spectra interpretation, duplexes that are useful in the invention assume a B-DNA type helical structure. It should be understood that it is not intended that the invention be limited by this belief and that the duplexes may, upon more conclusive analysis assume Z-DNA and/or A-DNA type helical structures.

These polynucleotide duplexes may be synthesized from native DNA or synthesized by chemical or recombinant techniques. Naturally occurring or recombinantly produced dsDNA of longer length may be digested (e.g., enzymatically, chemically and/or by mechanical shearing) and fractionated (e.g., by agarose gel or Sephadex™ column) to obtain polynucleotides of the desired length.

Alternatively, pairs of complementary single-stranded polynucleotide chains up to about 70 bases in length are readily prepared using commercially available DNA synthesizers and then annealed to form duplexes by conventional procedures. Synthetic dsDNA of longer length may be obtained by enzymatic extension (5′-phosphorylation followed by ligation) of the chemically produced shorter chains.

The polynucleotides may also be made by molecular cloning. For instance, polynucleotides of desired length and sequence are synthesized as above. These polynucleotides may be designed to have appropriate termini for ligation into specific restriction sites. Multiple iterations of these oligomers may be ligated in tandem to provide for multicopy replication. The resulting construct is inserted into a standard cloning vector and the vector is introduced into a suitable microorganism/cell by transformation. Transformants are identified by standard markers and are grown under conditions that favor DNA replication. The polynucleotides may be isolated from the other DNA of the cell/microorganism by treatment with restriction enzymes and conventional size fractionation (e.g., agarose gel, Sephadex™ column).

Alternatively, the polynucleotides may be replicated by the polymerase chain reaction (PCR) technology. Saiki et al (1985) Science 230:1350-1354; Saiki et al. (1988) Science 239:487-491; Sambrook et al. (1989) p 14.1-14.35.

In one embodiment, the polynucleotides are conjugated to a chemically-defined valency platform molecule in a manner that preserves their antibody binding activity. This is done, for example, by conjugating the polynucleotide to the valency platform molecule at a predetermined site on the polynucleotide chain such that the polynucleotide forms a pendant chain of at least about 20 base pairs measured from the conjugating site to the free (unattached) end of the chain.

In one embodiment, the polynucleotide duplexes are substantially homogenous in length and one strand of the duplex is conjugated to the carrier or valency platform molecule either directly or via a linker molecule. Synthetic polynucleotides may be coupled to a linker molecule before being conjugated to a carrier or valency platform molecule. Usually the linker containing strand of the duplex is coupled at or proximate (i.e., within about 5 base pairs) to one of its ends such that each strand forms a pendant chain of at least about 20 base pairs measured from the site of attachment of the strand to the linker molecule. The second strand is then annealed to the first strand to form a duplex. Thus, a conjugate within the present invention may be generally described by the following formula: [(PN)n-linker]m-valency platform molecule wherein PN=a double-stranded polynucleotide with “n” nucleotides, wherein n=at least about 20 and m=2-8. In other embodiments, n may have lower values.

In one embodiment, the polynucleotides of the conjugates are coupled to a linker molecule at or proximate one of their ends. The linker molecule is then coupled to the carrier or valency platform molecule. As described in U.S. Pat. No. 5,552,391 and incorporated herein by reference, exemplary of suitable linker molecules within the present invention are 6 carbon thiols such as HAD, a thio-6 carbon chain phosphate, and HADp S, a thio-6 carbon chain phosphorothioate. Chemically-defined valency platform molecules within the present invention are formed, for example, by reacting amino modified-PEG with 3,5-bis-(iodoacetamido) benzoyl chloride (hereinafter “IA-DABA”); 3-carboxypropionamide-N,N-bis-[(6′-N′-carbobenzyloxyaminohexyl)acetamide] 4“-nitrophenyl ester (hereinafter “BAHA”); 3-carboxypropionamide-N,N-bis-[(8′-N′-carbobenzyloxyamino-3′,6′-dioxaoctyl )acetamide] 4”-nitrophenyl ester (hereinafter “BAHAox”); or by reacting PEG-bis-chloroformate with N,N-di(2-[6′-N′-carbobenzyloxyaminohexanoamido]ethyl)amine (hereinafter “AHAB”) to form chemically-defined valency platform molecules.

For example, a defined double-stranded polynucleotide (PN) can be conjugated to a valency platform molecule by first providing a single chain consisting of approximately 20 alternating cytosine (C) and adenosine (A) nucleotides. Four CA chains may then be covalently conjugated through linkers such as HAD to four reactive sites on a derivatized platform molecule such as triethylene glycol. The valency platform molecule is synthesized to include groups such as bromoacetyl. During the conjugation, a leaving group is displaced by sulfur. A second single nucleotide chain consisting of approximately 20 alternating thymidine (T) and guanosine (G) nucleotides can then be annealed to the CA strand to form a double-stranded PN conjugate of the formula, [(PN)20-linker]4-valency platform molecule.

Alternatively, in another embodiment, the polynucleotide may be coupled to the derivatized valency platform molecule at the 3′ end of the polynucleotide via a morpholino bridge formed by condensing an oxidized 3′ terminal ribose on one of the strands of the polynucleotide with a free amino group on the derivatized platform molecule and then subjecting the adduct to reducing conditions to form the morpholino linkage, as described in U.S. Pat. No. 5,553,391. Such coupling requires the derivatized platform molecule to have at least an equal number of amino groups as the number of polynucleotide duplexes to be bound to the platform molecule. The synthesis of such a conjugate is carried out in two steps. The first step is coupling one strand of the polynucleotide duplex to the derivatized platform molecule via a condensation/reduction reaction. The oxidized 3′ terminal ribose is formed on the single polynucleotide strand by treating the strand with periodate to convert the 3′ terminal ribose group to an oxidized ribose group. The single-stranded polynucleotide is then added slowly to an aqueous solution of the derivatized platform molecule with a pH of about 6.0 to 8.0 at 2-8° C., generally with a reducing agent (such as sodium borohydride).

The molar ratio of polynucleotide to platform molecule in all the conjugation strategies will normally be in the range of about 2:1 to about 30:1, usually about 2:1 to about 8:1 and preferably about 4:1 to 6:1. In this regard, it is preferable that the conjugate not have an excessively large molecular weight as large molecules, particularly those with repeating units, of m.w. >200,000 may be T-independent immunogens. See Dintzis et al. (1983) J. Immunol. 131:2196 and Dintzis et al. (1989) J. Immunol. 143:1239. During or after the condensation reaction (normally a reaction time of 24 to 48 hr), a strong reducing agent, such as sodium cyanoborohydride, is added to form the morpholino group. The complementary strand of the duplex is then added to the conjugate and the mixture is heated and slowly cooled to cause the strands to anneal. The conjugate may be purified by gel permeation chromatography.

An alternative to the ribose strategy is forming aldehyde functionalities on the polynucleotides and using those functionalities to couple the polynucleotide to the carrier or platform molecule via reactive functional groups thereon. Advantage may be taken of the fact that gem vicinal diols, attached to the 3′ or 5′ end of the polynucleotide, may be oxidized with sodium periodate to yield aldehydes which can condense with functional amino groups of the platform molecule. When the diols are in a ring system, e.g., a five-membered ring, the resulting condensation product is a heterocyclic ring containing nitrogen, e.g., a six-membered morpholino or piperidino ring. The imino-condensation product is stabilized by reduction with a suitable reducing agent; e.g., sodium borohydride or sodium cyanoborohydride. When the diol is acyclic, the resulting oxidation product contains just one aldehyde and the condensation product is a secondary amine.

Another procedure involves introducing alkylamino or alkylsulfhydryl moieties into either the 3′ or 5′ ends of the polynucleotide by appropriate nucleotide chemistry, e.g., phosphoramidite chemistry. The nucleophilic groups may then be used to react with a large excess of homobifunctional cross-linking reagent, e.g., dimethyl suberimidate, in the case of alkylamine derivatives, or an excess of heterobifunctional cross-linking reagent, e.g., m-maleimidobenzoyl-N-hydroxysuccinimide ester (MBS) or succinimidyl (4-iodoacetyl) aminobenzoate (SIAB), for the alkylsulfhydryl derivatives. Once excess cross-linker is removed, the polynucleotide derivatives are reacted with amino groups on the platform molecule. Alternatively, the sulfhydryl group may be reacted with an electrophilic center on the platform, such as a maleimide or u-haloacetyl group or other appropriate Michael acceptor.

Still another strategy employs modified nucleosides. Suitable deoxynucleoside derivatives can be incorporated, by standard DNA synthetic chemistry, at desired positions in the polynucleotide, preferably on the 5′ or 3′ ends. These nucleoside derivatives may then react specifically and directly with alkylamino groups on the carrier or platform molecule. Alternatively, side reactions seen with the above-described dialdehyde chemistry, such as amine catalyzed beta-elimination, can be circumvented by employing appropriate nucleoside derivatives as the 3′ terminus of the chain to be attached. An example of this is 5′ methylene extension of ribose; i.e., a 5′ (2-hydroxyethyl)-group instead of a 5′ hydroxymethyl group. An alternative would be to use a phosphonate or phosphinate linkage for the 3′ terminal dinucleotide of the polynucleotide to be attached to the carrier or platform molecule.

A description of the synthesis of the conjugate LJP 394, a tetravalent conjugate, is described in Jones et al. (1995) and in U.S. Pat. No. 5,552,391, which are hereby incorporated by reference. LJP 394 comprises four 20-mer oligonucleotides consisting of alternating C and A nucleotides, (CA)10, attached to a platform and annealed with complementary 20-mer oligonucleotides consisting of alternating G and T nucleotides, (GT)10, oligonucleotide. The valency platform molecule used in LJP 394 is shown immediately below. In one embodiment, the platform molecule is 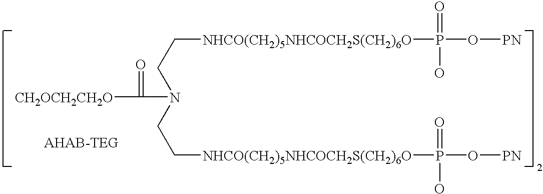
wherein PN is the polynucleotide. Accordingly, the epitope-presenting valency platform molecule administered to individuals with SLE in any of the methods of the invention described herein is LJP394 (also referred to as “Riquent”™), which comprises a molecule of the following formula: 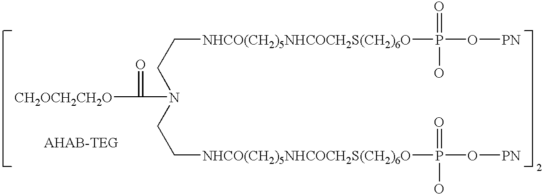
wherein PN is (CA)₁₀.(TG)₁₀

If a polypeptide (including peptide) is to be linked, standard chemical methods can be used. For example, U.S. Pat. Nos. 5,874,409, 6,410,775, 6,207,160, PCT WO97/46251, U.S. Ser. No. 09/328,199, and PCT WO99/64595 describe such linkages.

VII. Kits for Effecting Treatment and Monitoring Treatment

The invention also provides kits for effecting treatment using the methods of the present invention. The kits comprise an epitope described herein, optionally in the form of an epitope-presenting carrier, optionally in the form of an epitope-presenting valency platform molecule. In one embodiment, the kit comprises a pharmaceutical composition comprising (i) an epitope, optionally in the form of an epitope-presenting carrier such as an epitope-presenting valency platform molecule, and (ii) a pharmaceutically acceptable excipient. In one embodiment, the kits further comprise suitable packaging and/or instructions for use of the epitope, or pharmaceutical composition thereof, in accordance with the methods of treatment described herein. The instructions included in the kit may include, but are not necessarily limited to, instructions describing the administration of the epitope, or pharmaceutical composition thereof, to an individual to maintain sustained reduction of anti-dsDNA antibody of at least about 10% below baseline of the individual. Optionally, the instructions comprise a description of selecting an individual suitable for treatment with the epitope, or pharmaceutical composition thereof, based on identifying whether that individual has SLE and renal disease (including significant renal impairment) (as indicated by any clinical indicia described herein and/or known in the art), and preferably also further describe administration of the pharmaceutical composition for treatment of renal SLE. In some embodiments, the instructions comprise description of administering a conjugate to an individual having SLE who has renal disease (including significantly impaired renal function) (which may also describe one or more criteria for determining whether an individual having, or suspected of having lupus nephritis has renal impairment). In some embodiments, the kits further comprise one or more compositions for measuring level of renal function in an individual.

The invention also provides kits for monitoring a treatment of renal SLE and identifying likelihood of success of treatment for renal flare (which comprises an agent for reducing level of circulating anti-dsDNA antibody in an individual described herein), comprising materials for measuring the level of circulating anti-dsDNA antibody, and instructions for use in accordance with the methods of the invention.

In some embodiments, the kits may also contain supplies and instructions for measuring antibody affinities for use in the methods described herein, particularly affinity for an epitope which binds to anti-dsDNA antibodies. Accordingly, the kits of such embodiments contain (i.e., comprise) one or more dsDNA epitopes, preferably polynucleotides (preferably, double stranded (ds) DNA molecules) comprising an epitope which binds to an anti-dsDNA antibody from an individual (and the epitope-containing polynucleotide binds to an anti-dsDNA antibody from an individual). Accordingly, the kits comprise a molecule or moiety comprising a dsDNA epitope, such as any described herein. In one embodiment, the kit comprises a polynucleotide with (comprising) the sequence (or, alternatively, consisting essentially of or consisting of the sequence) 5′-GTGTGTGTGTGTGTGTGTGT-3′ (SEQ ID NO:1) (with or without its complement). In certain embodiments the dsDNA epitopes are not part of a conjugate with a non-immunogenic valency platform molecule. In other embodiments, the kits comprise the conjugates described herein, with instructions for using the conjugate to detect affinity of an individual's anti-dsDNA antibodies for the conjugate. Preferably, the conjugate is LJP 394.

In those embodiments containing materials and instructions for measurement of antibody affinity, such materials may be used, for example, to test an individual to determine if the individual is suitable or unsuitable for treatment with the conjugate(s), as well as for monitoring purposes. The affinity testing materials may also be used in determining affinity cut-off values (i.e., affinity values which correlate with clinical results).

The kits of this invention are in suitable packaging. Suitable packaging for epitope presenting conjugates includes, but is not limited to, vials, bottles, jars, flexible packaging (e.g., sealed Mylar or plastic bags), and the like.

Kits may optionally provide additional components such as, buffers and instructions for determining affinity or binding to anti-dsDNA antibody, such as capture reagents, developing reagents, labels, reacting surfaces, means for detection, control samples, and interpretive information. The instructions relating to measurement of antibody affinity may be for any measurement of antibody affinity, including, but not limited to, those assays described herein. Accordingly, in some embodiments, the instructions are for determining affinity using surface plasmon resonance. In other embodiments, the instruction are for determining affinity using direct binding assays and/or Farr assays. In some embodiments, reagents described above are supplied such that multiple measurements may be made, such as allowing for measurements in the same individual over time or multiple individuals.

In those embodiments comprising materials for testing antibody affinity, the dsDNA epitope(s) of the kit, preferably a polynucleotide(s) of the kit (whether in free form or attached to a conjugate or other matrix), generally contains, or alternatively consists of, the epitope that will be or is used in treatment, or has been demonstrated to have about the same affinity for an individual's anti-dsDNA antibodies as the epitope(s) that will be used in treatment. In other embodiments, the kits comprising a dsDNA epitope whose affinity for anti-dsDNA antibodies mimics or alternatively can be correlated to that of the dsDNA epitope to be used in treatment, such as 5′-GTGTGTGTGTGTGTGTGTGT-3′ (SEQ ID NO:1). These dsDNA epitopes can be used as “proxies” for the dsDNA epitope to be used in treatment, such as LJP 394, in assessing antibody affinity for the methods described herein.

Embodiments including materials for testing antibody affinity may comprise any appropriate means for detecting binding of the antibodies, such as a labeled anti-human antibody, when the presence of human anti-dsDNA antibodies is tested, wherein the label may be an enzyme, fluorophore, chemiluminescent material radioisotope or coenzyme. Generally, the label used will be an enzyme. Accordingly, in some embodiments, the kit(s) of the invention further comprises a label. In some embodiments, the polynucleotide in the kit(s) is conjugated to biotin. In a preferred embodiment, the dsDNA epitope (such as a polynucleotide, for example, double stranded DNA) is biotinylated. Biotinylation may also be accomplished using commercially available reagents (i.e., Pharmacia; Uppsala, Sweden). In another preferred embodiment, the biotinylated dsDNA epitope comprises, consists essentially or, or consists of is 5′-GTGTGTGTGTGTGTGTGTGT-3′ (SEQ ID NO:1).

In other embodiments, the invention provides a kit comprising (a) an epitope presenting conjugate as described herein, such as LJP 394; and (b) a polynucleotide (or other dsDNA epitope) used in the conjugate, or, alternatively, a polynucleotide comprising the polynucleotide used in the conjugate (or a molecule or moiety comprising the epitope to be used in the conjugate). These kits also contain the instructions for practicing a method(s) of the invention, as described above. When used for affinity measurements, the conjugate and/or polynucleotide may be biotinylated. In some embodiments, the kit contains instructions for administering the conjugate to an individual as well as instructions for using the conjugate and/or the polynucleotide (including a polynucleotide comprising the polynucleotide used in the conjugate) for detecting affinity for an antibody in an individual which binds to dsDNA as described herein. As discussed herein, a combination of a conjugate to be used for treatment and a molecule comprising a dsDNA epitope, the binding activity or affinity of which mimics, or can be correlated with, the epitope of the conjugates is used in the kits.

In related aspects, the invention provides articles of manufacture that comprise the contents of the kits described above. For instance, in one additional aspect, the invention provides an article of manufacture comprising a dsDNA epitope which specifically binds to an anti-dsDNA antibody, and instructions indicating use to treat SLE, including renal SLE, in the individual.

In other embodiments, the invention provides compositions (described herein) for use in any of the methods described herein, whether in the context of use as a medicament and/or use for manufacture of a medicament.

The following Examples are provided to illustrate, but not limit, the invention.

### EXAMPLES

### Example 1

SLE Patient Population Treated with LJP 394

A study was conducted in patients who met American College of Rheumatology criteria for the diagnosis of SLE, had a previous episode of SLE renal disease within four years, and had elevated anti-dsDNA≧15 IU/mL by the Farr assay at time of enrollment (Tan E M, et al. (1982) Arthritis Rheum 25:1271-7). Patients were excluded if they had evidence of a renal flare within three months of screening; were receiving prednisone or prednisone equivalent>20 mg/day, azathioprine>200 mg/day, methotrexate>25 mg/wk and/or cyclophosphamide at any dose within three months of screening; or a serum creatinine level≧2.5 mg/dL. The study was conducted in the U.S. and Europe according to Good Clinical Practices and all patients provided voluntary informed consent.

A pharmacodynamic assay has been developed to measure the binding affinity of patients' anti-dsDNA antibodies to LJP 394 (Sem D S, et al. (1999) Arch Biochem Biophys 372:62-8; McNeeley P A, et al. (2001) Lupus 10:526-532). The assay measures the binding of the total serum immunoglobulin G [IgG] fraction to the dsDNA epitope on LJP 394. Binding of IgG to the LJP 394 epitope is measured using surface plasmon resonance and the concentration required to reach half maximal binding is determined. This concentration defines the apparent K_{d}′ of the binding interaction and reflects the titer-weighted average affinity of the patient's IgG fraction for the LJP 394 epitope. Using this assay, patients were segregated into “high affinity” and “low affinity” subgroups. The segregation value was selected by comparing the affinity measured before exposure to LJP 394 with that following 16 weekly treatments with LJP 394 100 mg or placebo. The high-affinity (HA) population was defined as those patients with antibody binding affinities [K_{D}′]≦about 0.8 mg/mL pre-treatment.

### Example 2

Study Design for the Treatment of SLE Patients with LJP 394 (Phase II/III, 90-05)

In this double-blind, randomized controlled, multicenter trial, intravenously administered LJP 394 was compared with placebo in SLE patients with prior renal involvement. Patients were randomized to receive LJP 394 100 mg as a 2 ml bolus intravenous injection on a weekly basis or placebo for 76 weeks. After initiation of the trial, the protocol was amended to include 8 week off treatment periods alternating with 12 weekly treatments with 50 mg (1 ml bolus injection) LJP 394 or placebo. The first 16 weeks, when patients received 100 mg LJP 394 or placebo weekly, was considered the ‘induction period’, followed by ‘maintenance’, when patients alternated 8 off and 12 weeks on treatment. The 20-week cycles were to be repeated three times for a total of 60 weeks.

The primary endpoint was the time to a documented renal flare. A protocol-defined renal flare required that it be attributed to SLE by the treating physician and medical monitor. In addition, one or more of the following three criteria were required: 1) a reproducible increase in 24-hour urine protein levels to (a) >1,000 mg if the baseline value was <200 mg, (b) >2,000 mg if the baseline value was 200-1,000 mg, or (c) more than twice the value at baseline if the baseline value was >1,000 mg; 2) a reproducible increase in serum creatinine of >20% or at least 0.3 mg/dl, whichever was greater, accompanied by proteinuria (>1,000 mg/24 hours), hematuria (≧4 RBCs/high-power field), and/or RBC casts; or 3) new, reproducible hematuria (≧11-20 RBCs/high-power field) or a reproducible increase in hematuria by 2 grades compared with baseline, associated with >25% dysmorphic RBCs, glomerular in origin, exclusive of menses, accompanied by either an 800-mg increase in 24-hour urinary protein levels or new RBC casts. Secondary outcome measures included the number of renal flares, time to institution of therapy with high dose corticosteroids (HDCC) and/or cyclophosphamide, incidence of treatment with HDCC and/or cyclophosphamide, and number of major SLE flares. HDCC was defined as an increase in oral, intravenous or intramuscular prednisone (or prednisone equivalent) greater than or equal to 15 mg per day from baseline to a dose greater than 20 mg per day for more than two days or any dose exceeding 200 mg in a single day. Topical, intra-articular, intra-lesional, or intra-ocular corticosteroid administration was excluded. Details of Phase II/III trial is also described in Alarcon-Segovia D, et al. (2003) Arthritis Rheum. 48: 442-454.

### Example 3

Study Design for the Treatment of SLE Patients with LJP 394 (Phase III, 90-09)

Patients were treated with weekly doses of 100 mg of LJP 394 or with placebo. Patients were also permitted to receive certain other treatments including some but not all immunosuppressive drugs using a definition similar to Example 2. This randomized, double-blind, placebo-controlled study was conducted at more than 70 major medical centers in North America and Europe. Patients could remain in the study for up to 92 weeks.

The prospectively defined analysis groups were the intent-to-treat population and patients with impaired renal function. The intent-to-treat population was defined as patients with high-affinity antibodies to LJP 394. The patients with high-affinity antibodies to LJP 394 were those with a Kd′≦0.8 mg/ml. Patients with impaired renal function were defined as having a serum creatinine level of 1.5 mg/dL to 3.5 mg/dL at baseline. In general, patients with impaired renal function are at greater risk of progressing to renal flare, kidney failure, and dialysis.

The primary endpoint was time to renal flare. A renal flare was defined as a significant, reproducible increase in serum creatinine, urine protein or blood in the urine as described in Example 2. The secondary endpoint was time to treatment with HDCC. HDCC was defined as any dose of cyclophosphamide or an increase in prednisone of 15 mg/day or higher resulting in a final dose greater than 20 mg/day.

Other prospectively defined secondary outcomes included time to Major SLE flare, treatment associated maintenance and/or improvement in health-related quality of life, decreases in antibodies to dsDNA and associated increases in complement C3 levels. A Major SLE flare was defined as the occurrence of any one or more of the following due to manifestations of active SLE: treatment with HDCC or initiation or increase in treatment with other immunosuppressive agents, including azathioprine, mycophenolate mofetil, methotrexate, cyclosporin and leflunomide; or hospitalization or death. This definition of Major SLE flare was designed to capture serious events where patients were treated for hospitalization or death could have preceded the occurrence of a documented renal flare.

Complement changes were evaluated by determining the mean change from baseline in the complement protein C3 that indicates overall complement consumption due to active inflammation. Antibody changes were evaluated by determining the mean percent change of antibodies to dsDNA from baseline. Patients' assessments of disease activity and health-related quality of life were measured on a regular basis as well as at the time of, and 30 days following, a documented renal flare.

### Example 4

Increases in Anti-dsDNA Antibodies Correlate with Renal Flare

Two studies (Phase II/III or 90-05, and Phase III or 90-09) were conducted as described in Examples 2 and 3. Patients selection, duration of treatment, assay type were in these studies are further detailed in Table 1. All laboratory values were determined at a central laboratory. Baseline anti-dsDNA antibody levels were calculated as the mean of the last 2 determinations prior to initial administration of the study drug. Baseline values for all other laboratory measures were determined immediately before administration of the study drug. The upper limit of normal for the anti-dsDNA antibody assay at the central laboratory was 5 IU/ml.

TABLE 1
Key trial design considerations

| | Phase II/III | Phase III |
|---|---|---|
| Patients enrolled | 189 (High-affinity) | 298 |
| Protocol length | 18 month | 22 month |
| Average duration (LJP | 12.4 month, 12.7 month | 10.3 month, 11.4 |
| 394, Placebo) | | month |
| Assay type | Farr | Farr |
| Assay frequency | Weekly to monthly | Weekly to monthly |
| Assay QC | Central lab | Central lab |
| Positive anti-dsDNA | 189 | 298 |
| antibodies at baseline | | |

As shown in Table 2A, we observed that (1) increases in anti-dsDNA antibodies strongly correlate with incidence of renal flare; and (2) there is inverse correlation between the level of anti-dsDNA antibody and complement (C3) level.

TABLE 2A
Relationship between increased anti-dsDNA antibodies and renal flare

| | | | Phase |
|---|---|---|---|
| Variable | Observation | Phase III (90-09) | II/III (90-05) |
| Anti-ds DNA and | Increases in anti- | p < 0.0001 | p = 0.0007 |
| renal flare | dsDNA correlate | | |
| | with renal flare¹ | | |
| Anti-dsDNA and | Inverse correlation | p < 0.001 | p < 0.001 |
| C3 | between anti- | (Correlation | (Correlation |
| | dsDNA and C3² | coefficient: | coefficient: |
| | | −0.39) | −0.32) |
| C3 and renal flare | Decreases in C3 | p = 0.02 | Not |
| | correlate with renal | | significant |
| | flare¹ | | |

¹Cox regression with time-dependent covariate
²Based on Pearson Correlation for log-transformed anti-dsDNA data

As shown in Table 2B, patients with sustained increases in anti-dsDNA antibodies appear to be at greater risk of renal flare (p<0.0001). Thirty-three of 41 (81%) renal flares were observed in patients with sustained increases in anti-dsDNA. Patients with stable or decreasing anti-dsDNA are at less risk of renal flare, and 8 of 41 (20%) renal flares were observed in this group. The data show that the risk of renal flare was increased in patients with sustained increases in anti-dsDNA, independent of treatment assignment. Flare risk was increased whenever a patient was characterized as having a sustained increase in anti-dsDNA antibodies, independent of whether the increase occurred in the LJP 394 or placebo treatment group. Treatment with LJP 394 prevented patients from having sustained increases (i.e., decreased incidence) in anti-dsDNA antibodies when compared to placebo (p<0.0001 by Fisher's exact test).

TABLE 2B
Analysis of sustained increases in anti-dsDNA antibodies in LJP 394
treated and placebo group in Phase III trial

| | LJP 394 | | placebo | |
|---|---|---|---|---|
| | | # Renal | # | # Renal |
| | # Patients | Flares | Patients | Flares |
| Total Patients | 145 | 17 (12%) | 153 | 24 |
| Sustained increase in anti- | 41 | 12 (29%) | 84 | 21 (25%) |
| dsDNA antibodies¹ | | | | |
| Other² | 104 | 5 (5%) | 69 | 3 (4%) |
| p value³ | 0.0001 | | | 0.0006 |

¹Sustained increase ≧10% increase in anti-dsDNA for ≧⅔ of all observed values prior to HDCC or last visit.
²Other: any patients not meeting criteria for sustained increase.
³Fisher's exact test to determine if patients with sustained increases in anti-dsDNA antibodies are at increased risk of renal flare.

### Example 5

Patients with Sustained Reductions in Anti-dsDNA have Fewer Renal Flares

Patients in the studies described in the above examples were tested for levels of circulating anti-dsDNA antibodies in serum. Based on the levels of anti-dsDNA antibodies were segregated into two groups: sustained reduction group and other group. Patients that had sustained reduction were patients having at least about 10% reduction below baseline in anti-dsDNA antibody greater than or equal to ⅔ of all observed values prior to HDCC or last (most recent) dose of LJP 394 or placebo (the percent CV (co-efficient of variance) for the Farr assay is about 10%). Patients in the other group were any patients that did not meet the above criterion for sustained reduction.

The incidences of renal flares in patients of both sustained reduction group and the other group were statistically analyzed and the results are shown in Table 3 for Phase III and Table 4 for Phase II/III. We observed that renal flares were less likely in patients in the sustained reduction group when compared to patients in the other group as demonstrated in Table 3 (5/41 or 12% of total renal flares were in the sustained reduction group) and Table 4 (2/28 or 7% of total renal flares were in the sustained reduction group). Thus, patients with sustained reductions in antibodies to dsDNA had an 80% (p<0.0001, Fisher's exact test) and 86% (p<0.0004, Fisher's exact test) reduction in the risk of renal flare in the Phase III and the Phase II/III trials respectively compared with patients that did not have sustained reductions. The majority of renal flares were observed in patients that did not meet the criterion for having a sustained reduction in anti-dsDNA antibodies. This group included 36 of 41 (88%) total renal flares in Phase III and 26 of 28 (93%) total renal flares in Phase II/III. Treatment with LJP 394 resulted in higher frequency of patients with sustained reductions in anti-dsDNA antibodies (p<0.0001 by Fisher's exact test for Phase III trial and for Phase II/III trial). In Phase III trial, twice as many LJP 394-treated patients had sustained reductions compared with placebo-treated patients, while in Phase II/III, four times as many LJP 394-treated patients had sustained reductions compared with placebo-treated patients. Importantly, the risk of renal flare was reduced in patients with sustained reductions in anti-dsDNA antibodies, independent of treatment assignment. Renal flare risk was reduced in the group of patients who experienced a sustained reduction in anti-dsDNA antibody levels, independent of whether the reduction was occurred in the LJP 394-treatment group or the placebo-treatment group.

TABLE 3
In Phase III, patients with sustained reductions in anti-dsDNA
had fewer renal flares

| | Sustained | | | | |
|---|---|---|---|---|---|
| | reduction | | Other | | |
| | | # Renal | | # Renal | |
| | # Patients | flares | # Patients | flares | p value* |
| LJP 394 | 80 | 3 (4%) | 65 | 14 (22%) | |
| Placebo | 41 | 2 (5%) | 112 | 22 (20%) | |
| Total* | 121 | 5 (4%) | 177 | 36 (20%) | <0.0001 |
| LJP 394 | 54 | 1 (2%) | 38 | 6 (16%) | |
| Placebo | 13 | 1 (8%) | 84 | 20 (24%) | |
| Total* | 67 | 2 (3%) | 122 | 26 (21%) | 0.0004 |

*Fisher's exact test

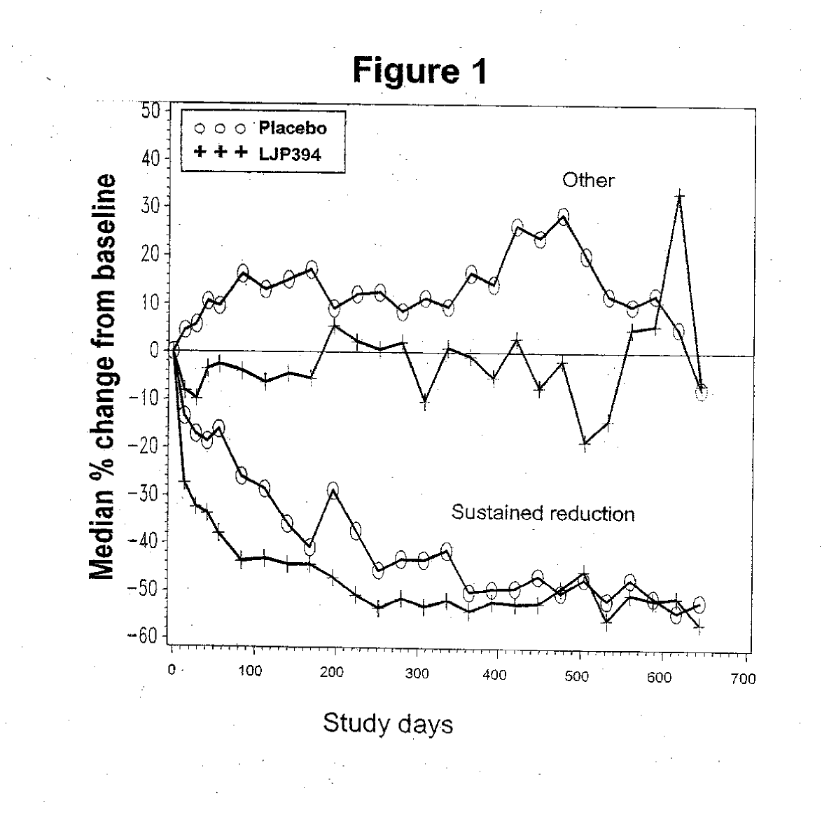

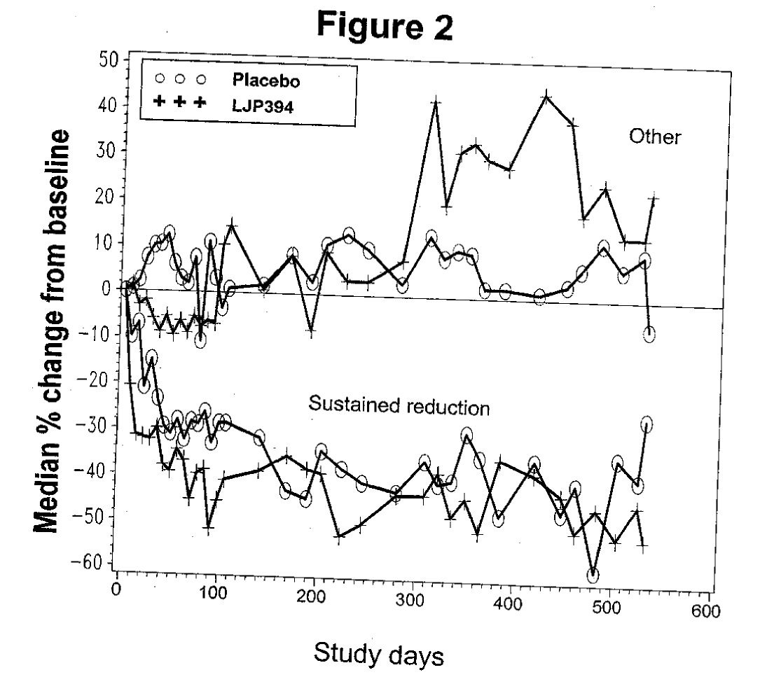

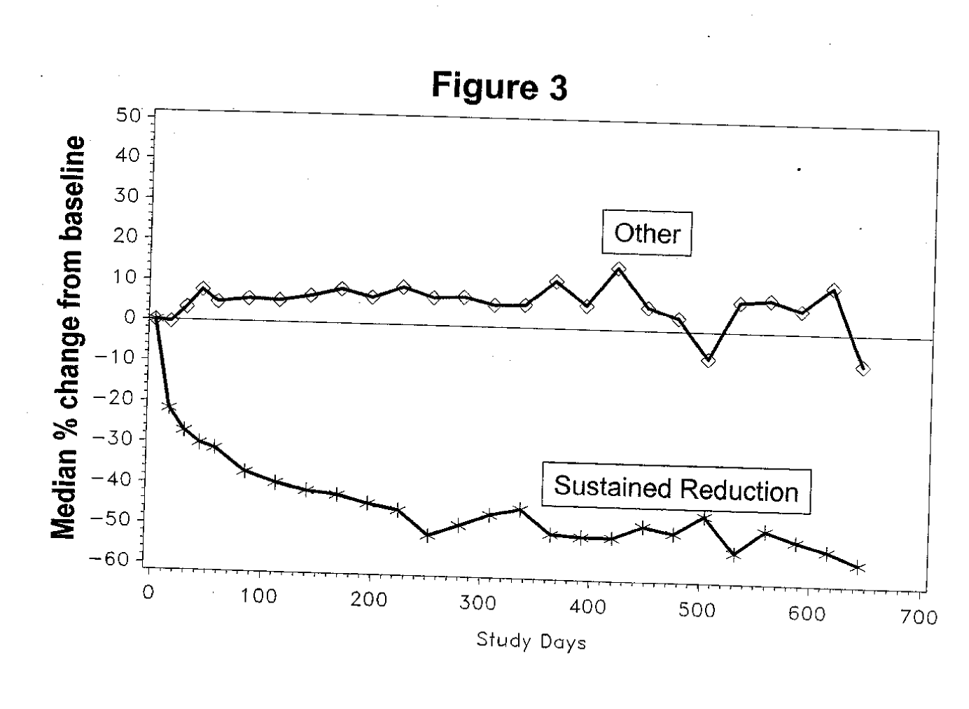

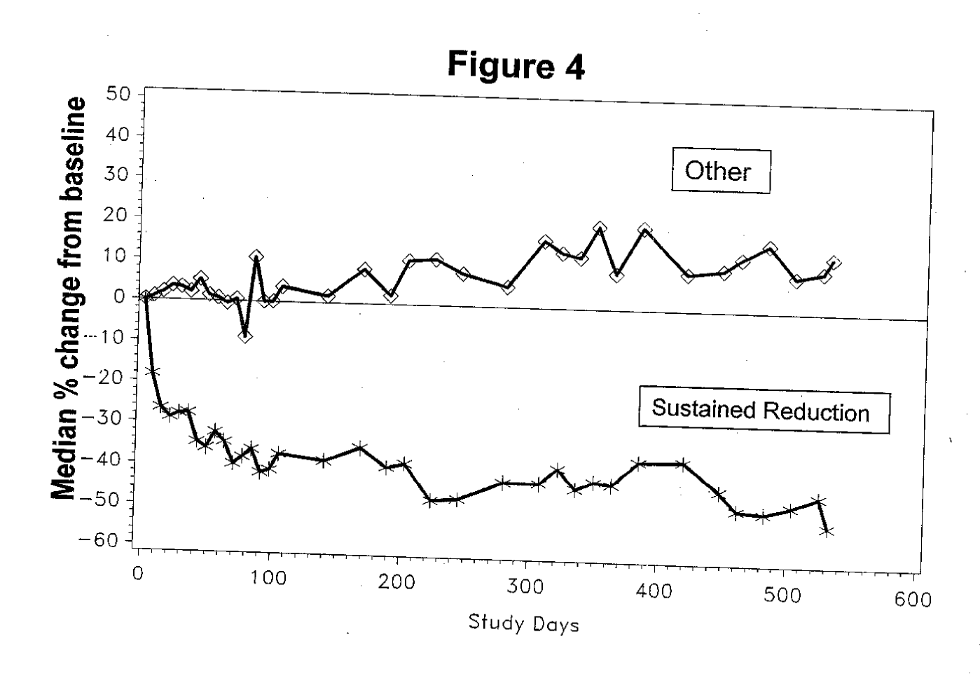

The levels of antibodies to dsDNA in placebo and LJP 394 treated group were determined for both Phase II/III and Phase III trial. The median percentage change of antibodies to dsDNA from baseline in the sustained reduction group and in the other group for both trials are shown in FIG. 1 (Phase III) and FIG. 2 (Phase II/III) with placebo and LJP 394 treated group shown separately and FIG. 3 (Phase III) and FIG. 4 (Phase II/III) with placebo and LJP 394 treated group combined.

### Example 6

Patients with Sustained Reductions in Anti-dsDNA have Fewer Major SLE Flares

The incidences of Major SLE flares in patients as in Example 5 of both sustained reduction group and the other group were also statistically analyzed and the results are shown in Table 5 for Phase III and Table 6 for Phase II/III.

TABLE 5
Incidence of Major SLE flare in patients with sustained
reduction in anti-dsDNA antibodies in Phase III trial

| | Sustained | | | | |
|---|---|---|---|---|---|
| | Reduction | | Other | | Total |
| | | Major SLE | | Major SLE | Major SLE |
| | Patients | Flare (%) | Patients | Flare (%) | Flare |
| Placebo | 41 | 4 (10%) | 112 | 43 (38%) | 47 |
| LJP 394* | 80 | 10 (13%) | 65 | 25 (38%) | 35 |
| Total** | 121 | 14 (12%) | 177 | 68 (38%) | 82 |

*p < 0.0001 by Fisher's exact test to determine if LJP 394 treated patients were more likely to have sustained reductions in anti-dsDNA antibodies than placebo-treated patients.
**p < 0.0001 by Fisher's exact test to determine if patients with sustained reductions in anti-dsDNA antibodies are at reduced risk of Major SLE flare.

TABLE 6
Incidence of Major SLE flare in patients with sustained reduction
in anti-dsDNA antibodies in Phase II/III* trial

| | Sustained | | | | |
|---|---|---|---|---|---|
| | reduction | | Other | | Total |
| | | Major SLE | | Major SLE | Major SLE |
| | Patients | Flare (%) | Patients | Flare (%) | Flare |
| Placebo | 13 | 3 (23%) | 84 | 38 (45%) | 41 |
| LJP 394** | 54 | 25 (9%) | 38 | 17 (45%) | 22 |
| Total† | 67 | 8 (12%) | 122 | 55 (45%) | 63 |

*High-affinity population
**p < 0.0001 by Fisher's exact test to determine if LJP 394 treated patients were more likely to have sustained reductions in anti-dsDNA antibodies than placebo-treated patients.
†p < 0.0001 by Fisher's exact test to determine if patients with sustained reductions in anti-dsDNA antibodies are at reduced risk of Major SLE flare.

The results in Table 5 and Table 6 indicated patients with sustained reductions in anti-dsDNA antibodies had a lower risk of Major SLE flare (p<0.0001 for Phase III and for Phase II/III; Fisher's exact test). Patients with sustained reductions in antibodies to dsDNA had an 70% and 75% reduction in risk of Major SLE flare in Phase III and Phase II/III trial, respectively (p<0.0001 for both trials). Fourteen of 82 (17%) Major SLE flares were observed in this group in Phase III trial and 8 of 63 Major SLE flares were observed in this group in Phase II/III trial. The majority of Major SLE flares were observed in patients that did not meet the criterion for having a sustained reduction in anti-dsDNA antibodies. This group included 68 of 82 (83%) total Major SLE flares in Phase III trial and 55 of 63 (87%) total Major SLE flares in Phase II/III trial. Tables 5 and 6 also indicated that treatment with LJP 394 resulted in higher frequency of patients with sustained reductions in anti-dsDNA antibodies (p<0.000 1 by Fisher's exact test for Phase III trial and for Phase II/III trial). In addition, data in Tables 5 and 6 indicated that the risk of Major SLE flare was reduced in patients with sustained reductions in anti-dsDNA antibodies, independent of treatment assignment.

### Example 7

Patients with Sustained Reduction in Anti-dsDNA Antibodies have Fewer Hospitalizations Including SLE Related Hospitalizations

The incidences of hospitalization and SLE related hospitalization in patients as in Example 5 of both sustained reduction group and the other group were also statistically analyzed and the results are shown in Tables 7 and 8 for Phase III and Tables 9 and 10 for Phase II/III. Hospitalization and SLE related hospitalization were determined by attending physician.

TABLE 7
Incidence of hospitalization in patients with sustained reduction
in anti-dsDNA antibodies in Phase III trial

| | Sustained | | | | |
|---|---|---|---|---|---|
| | reduction | | Other | | Total |
| | Patients | Hosp. | Patients | Hosp. | Hosp. |
| Placebo | 41 | 4 (10%) | 112 | 28 (25%) | 32 |
| LJP 394 | 80 | 8 (10%) | 65 | 19 (29%) | 27 |
| Total | 121 | 12 (10%) | 177 | 47 (27%) | 59 |

p value = 0.0003*
*Fisher's exact test
Sustained reduction - greater than or equal to 10% reduction in anti-dsDNA for greater than or equal to ⅔ of all observed values

Other—patients not included in sustained reduction definition

TABLE 8
Incidence of SLE related hospitalization in patients with
sustained reduction in anti-dsDNA antibodies in Phase III trial

| | | |
|---|---|---|
| Sustained | | |
| reduction | Other | Total |

| | Patients | Hosp. | Patients | Hosp. | Hosp. |
|---|---|---|---|---|---|
| Placebo | 41 | 2 (5%) | 112 | 13 (12%) | 15 |
| LJP 394 | 80 | 4 (5%) | 65 | 9 (14%) | 13 |
| Total | 121 | 6 (5%) | 177 | 22 (12%) | 28 |

p value = 0.0417*
*Fisher's exact test
Sustained reduction - greater than or equal to 10% reduction in anti-dsDNA for greater than or equal to ⅔ of all observed values
Other - patients not included in sustained reduction definition

TABLE 9
Incidence of hospitalization in patients with sustained reduction
in anti-dsDNA antibodies in Phase II/III trial

| | | |
|---|---|---|
| Sustained | | |
| reduction | Other | Total |

| | Patients | Hosp. | Patients | Hosp. | Hosp. |
|---|---|---|---|---|---|
| Placebo | 13 | 0 (0%) | 84 | 28 (33%) | 28 |
| LJP 394 | 54 | 5 (9%) | 38 | 11 (29%) | 16 |
| Total | 67 | 5 (7%) | 122 | 39 (32%) | 44 |

p value: 0.0001*
*Fisher's exact test
Sustained reduction - greater than or equal to 10% reduction in anti-dsDNA for greater than or equal to ⅔ of all observed values
Other - patients not included in sustained reduction definition

TABLE 10
Incidence of SLE related hospitalization in patients with sustained
reduction in anti-dsDNA antibodies in Phase II/III trial

| | | |
|---|---|---|
| Sustained | | |
| reduction | Other | Total |

| | Patients | Hosp. | Patients | Hosp. | Hosp. |
|---|---|---|---|---|---|
| Placebo | 13 | 0 (0%) | 84 | 19 (23%) | 19 |
| LJP 394 | 54 | 2 (4%) | 38 | 7 (18%) | 9 |
| Total | 67 | 2 (3%) | 122 | 26 (21%) | 28 |

p value: 0.0004*
*Fisher's exact test
Sustained reduction - greater than or equal to 10% reduction in anti-dsDNA for greater than or equal to ⅔ of all observed values
Other - patients not included in sustained reduction definition

As indicated in Tables 7-10, patients with sustained reductions in anti-dsDNA antibodies had fewer hospitalizations in Phase II/III trial (p=0.0001) and Phase III trial (p=0.0003) and fewer SLE related hospitalization in Phase II/III trial (p=0.0004) and in Phase III trial (p=0.0417).

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention.

We claim:

1. A method of treating systemic lupus erythematosus (SLE) in an individual, comprising administering to the individual an effective amount of an agent which reduces anti-dsDNA antibody in the individual, wherein the administration of the agent results in a sustained reduction of anti-dsDNA antibody, wherein the sustained reduction is at least about 10% below baseline in the individual, and wherein the individual is human.

2. The method of claim 1, wherein the agent comprises a dsDNA epitope which specifically binds to an anti-dsDNA antibody from the individual.

3. The method of claim 2, wherein the dsDNA epitope is a polynucleotide.

4. The method of claim 3, wherein the polynucleotide is DNA.

5. The method of claim 1, wherein the agent comprises a conjugate comprising a carrier and one or more double stranded DNA (dsDNA) epitopes, wherein the double stranded DNA epitopes are polynucleotides.

6. The method of claim 1, wherein the agent comprises a conjugate comprising a non-immunogenic valency platform molecule and two or more double stranded DNA (dsDNA) epitopes, wherein the double stranded DNA epitopes are polynucleotides.

7. The method of claim 5 or claim 6, wherein said polynucleotide comprises the sequence 5′-GTGTGTGTGTGTGTGTGTGT-3′ and its complement.

8. The method of claim 7, wherein the platform molecule is 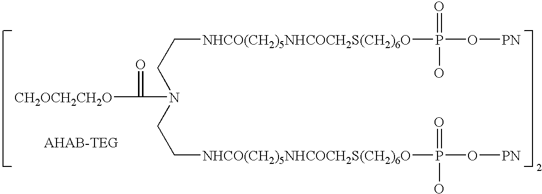
wherein PN is the polynucleotide.

9. The method of claim 7, wherein apparent equilibrium dissociation constant (K_{D}′) for the polynucleotide with respect to the antibody from the individual before or upon initiation of treatment is less than or equal to about 0.8 mg IgG per ml.

10. The method of claim 1, wherein the sustained reduction is at least about 20% below baseline in the individual.

11. The method of claim 1, wherein the sustained reduction is at least about 30% below baseline in the individual.

12. The method of claim 1, wherein the sustained reduction is for at least about four months.

13. The method of claim 1, wherein the sustained reduction is for at least about one year.

14. A method of reducing risk of renal flare in an individual with systemic lupus erythematosus, comprising reducing the levels of anti-dsDNA antibodies in the individual by administering an effective amount of an agent which reduces anti-dsDNA antibody in the individual, and maintaining sustained reduction of the anti-dsDNA antibodies, wherein the sustained reduction is at least about 10% below baseline in the individual, and wherein the individual is human.

15. The method of claim 14, wherein the agent comprises a dsDNA epitope which specifically binds to an anti-dsDNA antibody from the individual.

16. The method of claim 15, wherein the dsDNA epitope is a polynucleotide.

17. The method of claim 16, wherein the polynucleotide is DNA.

18. The method of claim 14, wherein the agent comprises a conjugate comprising a carrier and one or more double stranded DNA (dsDNA) epitopes, wherein the double stranded DNA epitopes are polynucleotides.

19. The method of claim 14, wherein the agent comprises a conjugate comprising a non-immunogenic valency platform molecule and two or more double stranded DNA (dsDNA) epitopes, wherein the double stranded DNA epitopes are polynucleotides.

20. The method of claim 18 or claim 19, wherein said polynucleotide comprises the sequence 5′-GTGTGTGTGTGTGTGTGTGT-3′ and its complement.

21. The method of claim 20, wherein the platform molecule is 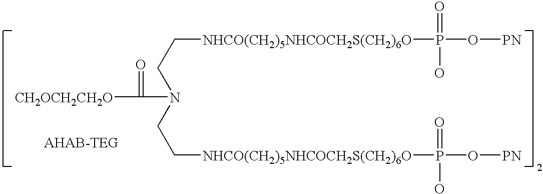
wherein PN is the polynucleotide.

22. The method of claim 20, wherein apparent equilibrium dissociation constant (K_{D}′) for the polynucleotide with respect to the antibody from the individual before or upon initiation of treatment is less than or equal to about 0.8 mg IgG per ml.

23. The method of claim 14, wherein the sustained reduction is at least about 20% below baseline in the individual.

24. The method of claim 14, wherein the sustained reduction is at least about 30% below baseline in the individual.

25. The method of claim 14, wherein the sustained reduction is for at least about four months.

26. The method of claim 14, wherein the sustained reduction is for at least about one year.


SEQUENCE LISTING (3 sequences)

SEQ ID NO: 1 - DNA, 20 bp, Artificial Sequence
  Synthetic Construct
tgtgtgtgtgtgtgtgtgtg

SEQ ID NO: 2 - DNA, 20 bp, Artificial Sequence
  Synthetic Construct
cacacacacacacacacaca

SEQ ID NO: 3 - protein, 5 aa, Artificial Sequence
  Synthetic Construct
  VARIANT 1, 3: Xaa = Asp or Glu
  VARIANT 5: Xaa = Ser or Gly
XWXYX